(19) United States
(12) Patent Application Publication
(10) Pub. No.: US 2026/0250257 A1
(43) Pub. Date: Aug. 27, 2026

(54) ANTIBACTERIAL AND SYNERGISTIC ACTIVE COMPOUND AND USE THEREOF

(71) Applicants: EAST CHINA UNIVERSITY OF SCIENCE AND TECHNOLOGY, Shanghai (CN); SHANGHAI JIAO TONG UNIVERSITY, Shanghai (CN)

(72) Inventors: Xiaoyong XU, Shanghai (CN); Daijie CHEN, Shanghai (CN); Zhong LI, Shanghai (CN); Yu YIN, Shanghai (CN); Yuce CHEN, Shanghai (CN); Ting LIN, Shanghai (CN)

(21) Appl. No.: 19/160,664

(22) PCT Filed: Feb. 28, 2024
(86) PCT No.: PCT/CN2024/079122

§ 371 Date: Aug. 28, 2025

(30) Foreign Application Priority Data
Feb. 28, 2023 (CN) 202310178218.5

(51) Int. Cl. C07D 277/40; A61K 31/426; A61K 31/427; A61K 31/437; A61K 31/4439; A61K 31/4709; A61K 31/506; A61K 38/12; A61P 31/04; C07D 417/04; C07D 417/10; C07D 417/12; C07D 471/04

(52) U.S. Cl. CPC C07D 277/40; A61K 31/426; A61K 31/427; A61K 31/437; A61K 31/4439; A61K 31/4709; A61K 31/506; A61K 38/12; A61P 31/04; C07D 417/04; C07D 417/10; C07D 417/12; C07D 471/04

(57) ABSTRACT

A compound as represented by formula I, or an optical isomer, a cis-trans isomer or a pharmaceutically acceptable salt thereof, and a preparation and the use thereof. The compound as represented by formula I can provide polymyxin a significant synergistic effect with regard to resisting various gram-negative bacteria, and can be applied to the treatment of infections caused by polymyxin-insensitive bacteria or drug-resistant bacteria.

### TECHNICAL FIELD

The present invention relates to the field of pharmacy. In particular, the present invention relates to guanidine compounds and the use of such guanidine compounds for enhancing the activity of polymyxins.

### BACKGROUND

### 1. Polymyxins

Since the advent of penicillin in the 1940s, various types of antibiotics have been developed, making tremendous contributions to the treatment of bacterial infections in humans. However, due to the increase in bacterial infections and the inappropriate use of antibiotics, clinically resistant bacterial strains have also emerged. The WHO has recently released a global priority list of antibiotic-resistant bacterial pathogens. Based on multiple criteria, such as mortality rate, prevalence of resistance, and treatability, these bacterial pathogens are categorized into critical, high, and medium priority levels. Among them, infections caused by multidrug-resistant (MDR), extensively drug-resistant (XDR), and pandrug-resistant (PDR) Gram-negative bacteria are particularly prominent. Among clinically isolated Gram-negative bacteria, the most frequently encountered include Escherichia coli (E. coli), Klebsiella pneumoniae, Acinetobacter baumannii and Pseudomonas aeruginosa. How to treat infections caused by these “superbugs” is becoming an increasingly challenging issue.

Polymyxin has been used clinically since 1952 and exhibits excellent bactericidal effects against Gram-negative bacteria. However, its use was limited by significant nephrotoxicity and neurotoxicity. With the advent of new carbapenem antibiotics, polymyxin gradually fell out of clinical use. Nevertheless, with the emergence of “superbugs” resistant to antibiotics, polymyxin had to be reintroduced as the “last line of defense” against Gram-negative bacterial infections in the 21^{st} century. It is particularly used for infections caused by extensively drug-resistant Acinetobacter baumannii, Klebsiella pneumoniae, and Escherichia coli, as these bacteria are currently only sensitive to polymyxin.

However, with the continued use of polymyxin, bacteria resistant to it have also emerged. Therefore, there is an urgent need in this field for technical means to address polymyxin resistance.

### SUMMARY OF THE INVENTION

The purpose of the present invention is to provide an antibiotic synergist capable of enhancing the activity of antibiotics, such as polymyxins, against various bacteria, for example Gram-negative bacteria, thereby enabling its application in the treatment of bacterial infections that are resistant to or insensitive to antibiotics.

Another purpose of the present invention is to provide a combination of the antibiotic synergist and an antibiotic, such as polymyxin.

Yet another purpose of the present invention is to provide a method for treating bacterial infections that are resistant to or insensitive to antibiotics, such as polymyxins.

In the first aspect, the present invention provides the use of a compound of Formula I, or an optical isomer, cis-trans isomer, or a pharmaceutically acceptable salt thereof, in the preparation of an antibiotic synergist:

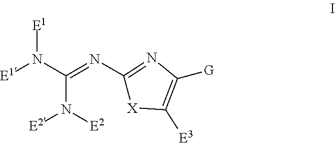

wherein:
E¹ and E^{1′} are each independently a hydrogen, acyl, substituted or unsubstituted C₁₋₆ alkyl, or substituted or unsubstituted C₁₋₆ alkoxy;
E² and E^{2′} are each independently a hydrogen, acyl, substituted or unsubstituted C₁₋₆ alkyl, or substituted or unsubstituted C₁₋₆ alkoxy; or
one of E¹ and E^{1′} is connected with one of E² and E^{2′} to form a substituted or unsubstituted C₁₋₆ alkylene;
E³ is a hydrogen, substituted or unsubstituted C₁₋₆ alkyl, substituted or unsubstituted C₁₋₆ alkoxy, substituted or unsubstituted C₃₋₇ cycloalkyl, or halogen;
X is NE⁴, O, S, or Se; E⁴ is a hydrogen, halogen, substituted or unsubstituted C₁₋₃ alkyl, or substituted or unsubstituted C₁₋₃ alkoxy;
G is a substituted or unsubstituted C₁₋₆ alkyl, substituted or unsubstituted C₂₋₆ alkenyl, substituted or unsubstituted C₂₋₆ alkynyl, substituted or unsubstituted C₃₋₇ cycloalkyl, substituted or unsubstituted C₅₋₇ cycloalkenyl, substituted or unsubstituted C₆₋₁₀ aryl (for example, phenyl or naphthyl), substituted or unsubstituted 5- or 6-membered heterocyclic group, or substituted or unsubstituted 8- to 14-membered heteroaryl bicyclic or tricyclic ring system; and the “substituted” refers to a substitution by one or more groups (for example, 1-4 or 1-3) selected from: a halogen, cyano, nitro, R′, OR′, Si(R′)₃, NR′R″, C(O)R′, C(O)OR′, C(O)NR′R″, SR′, S(O)ₘR¹, S(O)₂NR′R″, OC(O)R¹, OC(O)NR′R″, OS(O)₂R¹, OS(O)₂NR′R″, N(R²)C(O)R¹, N(R²)C(O)NR′R″, N(R²)S(O)₂R¹ or N(R²)S(O)₂NR′R″;
wherein R′ and R″ are each independently H, C₁₋₆ alkyl, C₁₋₆ haloalkyl, C₂₋₆ alkenyl, C₂₋₆ haloalkenyl, C₂₋₆ alkynyl, C₂₋₆ haloalkynyl, or substituted or unsubstituted C₃₋₇ cycloalkyl, substituted or unsubstituted C₅₋₇ cycloalkenyl, substituted or unsubstituted phenyl, substituted or unsubstituted naphthyl, substituted or unsubstituted 5- or 6-membered heterocyclyl, or substituted or unsubstituted 8- to 12-membered heteroaryl bicyclic ring system, and the “substituted” refers to a substitution by one or more (e.g., 1-4, 1-3, or 1-2) groups selected from: a halogen, cyano, nitro, C₁₋₆ alkyl, C₁₋₆ haloalkyl, C₂₋₆ alkenyl, C₂₋₆ haloalkenyl, C₂₋₆ alkynyl, C₂₋₆ haloalkynyl, hydroxy, hydroxy-C₁₄ alkyl, OR³, NR³R⁴, C(O)R³, C(O)OR³, C(O)NR³R⁴, SR³, S(O)ₘR⁵, S(O)₂NR³R⁴, OC(O)R⁵, OC(O)NR³R⁴, OS(O)₂R⁵, OS(O)₂NR³R⁴, N(R⁶)C(O)R⁵, N(R⁶)C(O)NR³R⁴, N(R⁶)S(O)₂R⁵ or N(R⁶)S(O)₂NR³R⁴;
R¹ is C₁₋₆ alkyl, C₁₋₆ haloalkyl, C₂₋₆ alkenyl, C₂₋₆ haloalkenyl, C₂₋₆ alkynyl, C₂₋₆ haloalkynyl, or substituted or unsubstituted C₃₋₇ cycloalkyl, substituted or unsubstituted C₅₋₇ cycloalkenyl, substituted or unsubstituted phenyl, substituted or unsubstituted naphthyl, substituted or unsubstituted 5- or 6-membered heterocyclyl, or substituted or unsubstituted 8- to 12-membered heteroaryl bicyclic ring system; and the “substituted” refers to a substitution by one or more groups selected from: a halogen, cyano, nitro, C₁₋₆ alkyl, C₁₋₆ haloalkyl, C₂₋₆ alkenyl, C₂₋₆ haloalkenyl, C₂₋₆ alkynyl, and C₂₋₆ haloalkynyl, OR³, NR³R⁴, C(O)R³, C(O)OR³, C(O)NR³R⁴, SR³, S(O)ₘR⁵, S(O)₂NR³R⁴, OC(O)R⁵, OC(O)NR³R⁴, OS(O)₂R⁵, OS(O)₂NR³R⁴, N(R⁶)C(O)R⁵, N(R⁶)C(O)NR³R⁴, N(R⁶)S(O)₂R⁵ or N(R⁶)S(O)₂NR³R⁴;
R² is H, C₁₋₆ alkyl, C₁₋₆ haloalkyl, C₂₋₆ alkenyl, C₂₋₆ haloalkenyl, C₂₋₆ alkynyl or C₂₋₆ haloalkynyl, or substituted or unsubstituted C₃₋₇ cycloalkyl, substituted or unsubstituted C₅₋₇ cycloalkenyl, substituted or unsubstituted phenyl, substituted or unsubstituted naphthyl, substituted or unsubstituted 5- or 6-membered heterocyclic group, or substituted or unsubstituted 8- to 12-membered heteroaryl bicyclic ring system; and the “substituted” refers to a substitution by one or more groups selected from: a halogen, cyano, nitro, C₁₋₆ alkyl, C₁₋₆ haloalkyl, C₂₋₆ alkenyl, C₂₋₆ haloalkenyl, C₂₋₆ alkynyl, C₂₋₆ haloalkynyl, OR³, NR³R⁴, C(O)R³, C(O)OR³, C(O)NR³R⁴, SR³, S(O)ₘR⁵, S(O)₂NR³R⁴, OC(O)R⁵, OC(O)NR³R⁴, OS(O)₂R⁵, OS(O)₂NR³R⁴, N(R⁶)C(O)R⁵, N(R⁶)C(O)NR³R⁴, N(R⁶)S(O)₂R⁵ or N(R⁶)S(O)₂NR³R⁴;
wherein R³, R⁴ and R⁶ are each independently H, C₁₋₆ alkyl, C₁₋₆ haloalkyl, C₂₋₆ alkenyl, C₂₋₆ haloalkenyl, C₂₋₆ alkynyl, or C₂₋₆ haloalkynyl;
R⁵ is C₁₋₆ alkyl, C₁₋₆ haloalkyl, C₂₋₆ alkenyl, C₂₋₆ haloalkenyl, C₂₋₆ alkynyl, or C₂₋₆ haloalkynyl;
and m is 1 or 2.

In a specific embodiment, the compound of Formula I is as shown in Formula I′:

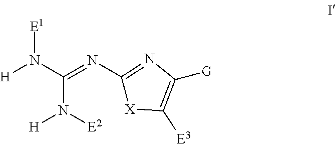

wherein E¹, E², E³, X and G are described as above.

In a specific embodiment, the compound of Formula I′ is as shown in Formula II-a or II-b:

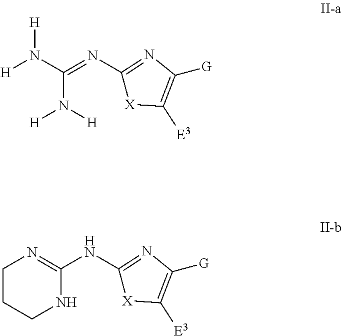

Wherein E³, X and G are described as above.

In a specific embodiment, the compound of Formula II-a or II-b is as shown in Formula III-a or III-b:

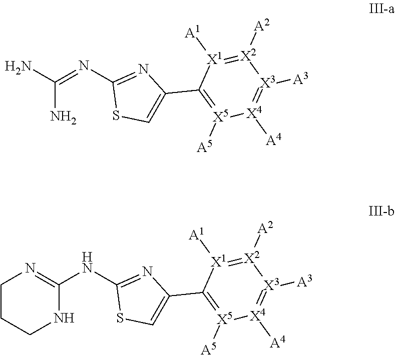

Wherein each of X¹, X², X³, X⁴ and X⁵ is independently selected from C or N;
each of A¹, A², A³, A⁴ and A⁵ is independently selected from H, substituted or unsubstituted C₁₋₆ alkyl, substituted or unsubstituted C₁₋₆ haloalkyl, substituted or unsubstituted C₂₋₆ alkenyl, substituted or unsubstituted C₂₋₆ haloalkenyl, substituted or unsubstituted C₂₋₆ alkynyl, substituted or unsubstituted C₂₋₆ haloalkynyl, substituted or unsubstituted C₃₋₇ cycloalkyl, substituted or unsubstituted C₅₋₇ cycloalkenyl, substituted or unsubstituted phenyl, substituted or unsubstituted 5- or 6-membered heterocyclyl; and the “substituted” is the same as those for G described above.

In a specific embodiment, the compound of Formula III-a or III-b is as shown in Formula IV-a or IV-b:

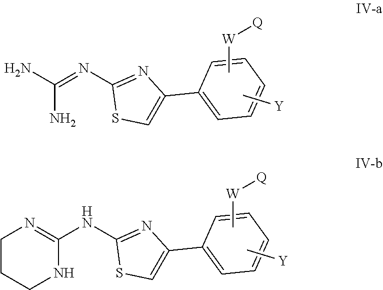

wherein W is selected from: —O(CH₂)ₚ—, —S(CH₂)ₚ—, —CH₂—, —CO—, —S(O)ₚ—, —C(O)NH—, —S(O)₂NH—, —NHC(O)—, Oxime ether group, Thiocarbonyl-NH—; preferably —O(CH₂)ₚ—, —S(CH₂)ₚ—;
p is 0, 1 or 2;
Q is selected from a substituted or unsubstituted C₁₋₆ alkyl, substituted or unsubstituted C₂₋₆ alkenyl, substituted or unsubstituted C₂₋₆ alkynyl, substituted or unsubstituted C₃₋₇ cycloalkyl, substituted or unsubstituted C₅₋₇ cycloalkenyl, substituted or unsubstituted phenyl, substituted or unsubstituted 5- or 6-membered heterocyclyl; and the “substituted” is the same as those for G described above;
Y is selected from a hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₇ alkylene, substituted or unsubstituted C₅₋₇ cycloalkenyl, substituted or unsubstituted phenyl, substituted or unsubstituted naphthyl, substituted or unsubstituted 5- or 6-membered heterocyclic group; and the “substituted” is the same as those for G described above.

In a specific embodiment, Q in the Formula III-a or III-b is as shown in Formula V:

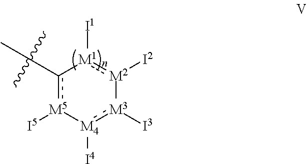

wherein M¹ is selected from C, CH, S, O or N;
each of M², M³, M⁴ and M⁵ is independently selected from: C, CsH, S, O, N;
n is an integer selected from 0-2;
each of I¹, I², I³, I⁴ and I⁵ is independently selected from a hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₇ alkylene, substituted or unsubstituted C₅₋₇ cycloalkenyl, substituted or unsubstituted phenyl, substituted or unsubstituted 5- or 6-membered heterocyclic group; and the “substituted” is the same as those described for G.

In a preferred embodiment, Q in formulas III-a and III-b is selected from following substituted or unsubstituted groups:

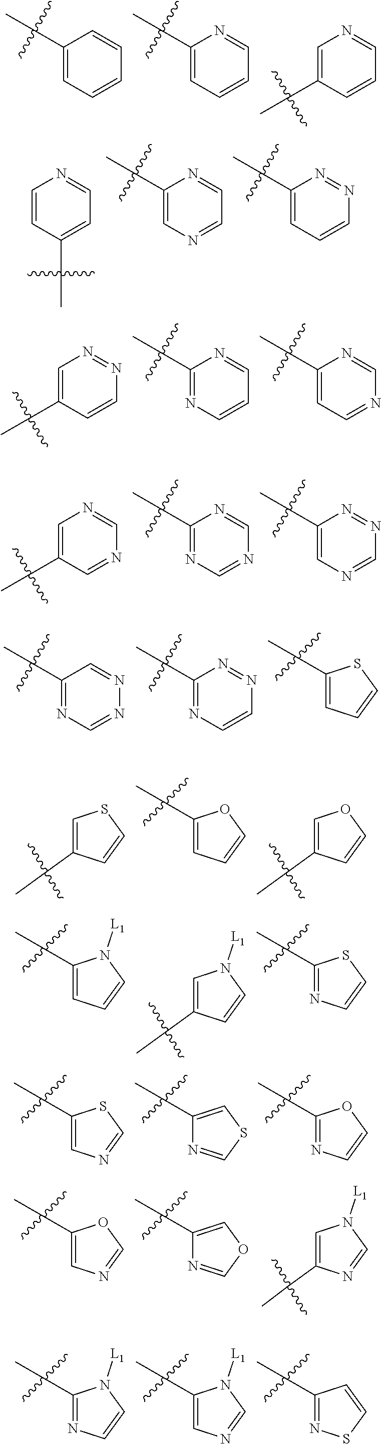

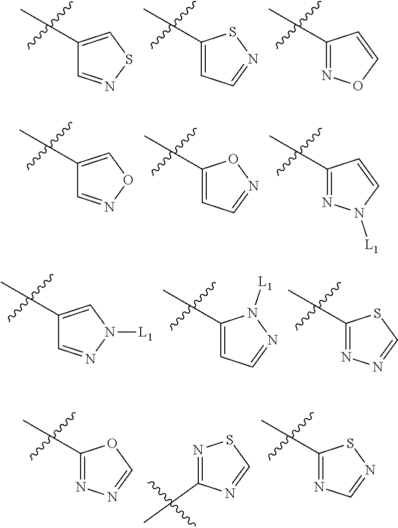

L¹ is selected from a substituted or unsubstituted C₂₋₄ ester group, substituted or unsubstituted amide group, substituted or unsubstituted C₁₋₁₂ (preferably C₁₋₆, more preferably C₁₋₄) alkyl or alkoxy, substituted or unsubstituted C₃₋₁₂ (preferably C₃₋₆) cycloalkyl, substituted or unsubstituted C₂₋₁₂ (preferably C₂₋₆, more preferably C₂₋₄) alkenyl or alkynyl, substituted or unsubstituted —CH₂—Z—(C₁₋₁₂ (preferably C₁₋₆, more preferably C₁₋₄) alkyl or C₃₋₁₂ (preferably C₃₋₆) cycloalkyl) (wherein Z is O, NH, or S), substituted or unsubstituted C₅₋₁₂ aryl or heteroaryl, substituted or unsubstituted —CH₂—(C₅₋₁₂ aryl or heteroaryl); and the “substituted” is the same as those for G described above.

In a preferred embodiment, Q in formulas III-a and III-b is selected from following groups:

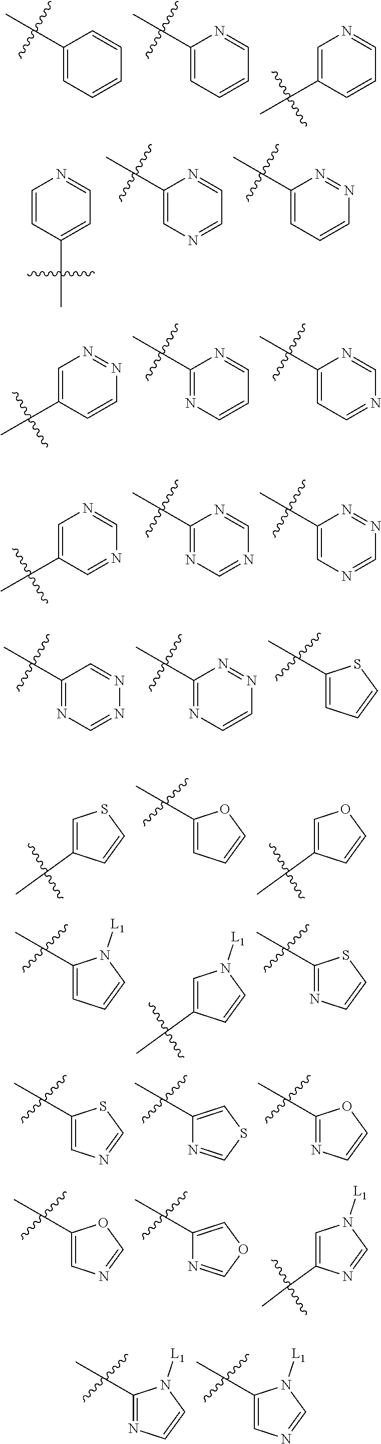

preferably, Q is selected from:

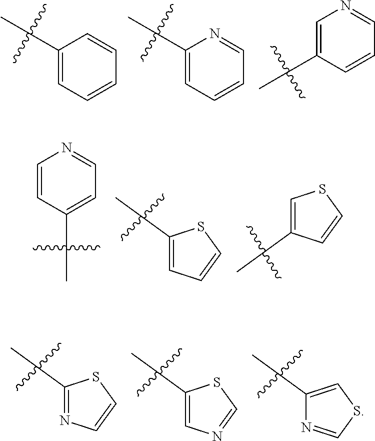

In a specific embodiment, in the compound:
E¹, E^{1′}, E² and E^{2′} are H; or
one of E¹ and E^{1′} is connected with one of E² and E^{2′} to form a substituted or unsubstituted C₂₋₄ alkylene;
E³ is a hydrogen, substituted or unsubstituted C₁₋₆ alkyl or halogen;
X is NH, O, S or Se;
G is selected from a substituted or unsubstituted phenyl, or substituted or unsubstituted 8- to 14-membered heteroaryl bicyclic ring system; and the “substituted” is described as above.

In a specific embodiment, the compound is any one of those numbered I-1 to I-200;
preferably, the compound is any one of those numbered as follows:
I-9, I-14, I-19, I-50, I-60, I-75, I-81, I-82, I-86, I-86, I-87, I-88, I-89, I-90, I-92, I-93, I-95, I-96, I-97, I-98, I-99, I-100, I-108, I-123, I-124, I-125, I-126, I-127, I-128, I-129, I-130, I-131, I-132, I-133, I-142, I-143, I-144, I-145, I-146, I-147, I-148, I-149, I-150, I-151, I-152, I-153, I-154, I-155, I-156, I-157, I-158, I-159, I-160, I-161, I-162, I-163, I-170, I-176.

In a specific embodiment, the antibiotic is an anti-Gram-negative bacterial antibiotic or a polypeptide antibiotic.

In a preferred embodiment, the antibiotic is selected from one or more of the following: polymyxin antibiotics, carbapenem antibiotics, aminoglycoside antibiotics, cephalosporin (such as second-, third-, or fourth-generation of cephalosporin antibiotics) antibiotics, quinolone antibiotics, bacitracin (including bacitracin and gramicidin), and vancomycin.

In a preferred embodiment, the antibiotic is a polymyxin antibiotic; and preferably, it is polymyxin B or polymyxin E.

In the second aspect, the present invention provides a pharmaceutical composition comprising:
a. the compound according to the first aspect, or an optical isomer, cis-trans isomer, or a pharmaceutically acceptable salt thereof, or a combination thereof; and
b. an antibiotic; and
c. optionally, a pharmaceutically acceptable carrier and/or excipient.

In a preferred embodiment, the dosage form of the composition is selected from: a tablet, lozenge, bean-shaped capsule, dispersion, suspension, solution, capsule, patch, or a combination thereof.

In a preferred embodiment, the antibiotic is an anti-Gram-negative bacterial antibiotic or a polypeptide antibiotic.

In a preferred embodiment, the antibiotic is selected from one or more of the following: polymyxin antibiotics, carbapenem antibiotics, aminoglycoside antibiotics, cephalosporin (e.g., 2^{nd}-, 3^{rd}-, or 4^{th}-generation cephalosporin antibiotics) antibiotics, quinolone antibiotics, bacitracin (bacitracin, gramicidin), and vancomycin.

In a preferred embodiment, the antibiotic is a polymyxin antibiotic; preferably, polymyxin B.

In a preferred embodiment, the antibiotic is polymyxin E.

In a preferred embodiment, the effective concentration of the compound is 0.01-150 mg/L.

In a preferred embodiment, the weight ratio of the compound of Formula I to the antibiotic is 1:50 to 200:1, preferably 1:10 to 100:1.

It should be understood that, within the scope of the present invention, the aforementioned technical features of the invention and the various technical features specifically described below (such as in the examples) can be combined with each other to form new or preferred technical solutions, which will not be repeated one by one herein due to the limited contents.

### MODES FOR CARRYING OUT THE INVENTION

After extensive and in-depth research, the inventors unexpectedly discovered a series of aryliminylthiazole compounds exhibiting excellent antibiotic (e.g., polymyxin) synergistic activity. Therefore, the compounds of the present invention can be used to treat bacterial infections, such as those caused by Escherichia coli, Acinetobacter baumannii, Klebsiella pneumoniae and Pseudomonas aeruginosa, particularly infections caused by strains of E. coli, A. baumannii, K pneumoniae and P. aeruginosa insensitive to antibiotics or showing weak antibacterial activity. The dosage of antibiotics, such as polymyxins, required during the treatment of bacterial infections can be effectively reduced by using the compounds of the present invention, thereby significantly lowering the risks imposed by the toxic side effects of antibiotics to the treated patients, based on which the present invention has been completed.

### Definition on Groups

The term “alkyl” as used herein has the meaning commonly understood by a skilled person, which refers to a group formed by removing one hydrogen atom from an alkane molecule. For example, the term “C₁₋₆ alkyl” refers to a straight-chain or branched alkyl group having 1 to 6 carbon atoms, such as a methyl, ethyl, propyl, isopropyl, butyl, isobutyl, sec-butyl, tert-butyl, or similar groups.

The term “alkenyl” as used herein has the meaning commonly understood by a skilled person. For example, the term “C₂₋₆ alkenyl” refers to a straight-chain or branched alkenyl group having 2 to 6 carbon atoms, such as a vinyl, allyl, 1-propenyl, isopropenyl, 1-butenyl, 2-butenyl, or similar groups.

The term “alkylene” as used herein has the meaning commonly understood by a skilled person, which refers to a group formed by removing two hydrogen atoms from an alkane molecule. The definitions of “alkenylene”, “alkynylene”, “cycloalkylene”, “cycloalkenylene”, “phenylene”, “naphthylene”, “heterocyclylene”, or “heteroarylbicyclic or tricyclic ring system” are analogous to that of “alkylene”.

The term “alkynyl” as used herein has the meaning commonly understood by a skilled person. For example, the term “C₂₋₆ alkynyl” refers to a straight-chain or branched alkynyl group having 2 to 6 carbon atoms, such as an ethynyl, propynyl, or similar groups.

The term “acyl” as used herein has the meaning commonly understood by a skilled person, and refers to the portion remaining after the hydroxyl group is removed from a carboxyl (R—C(═O)—OH), leaving a carbonyl (C═O).

The term “cycloalkyl” as used herein has the meaning commonly understood by a skilled person. For example, the term “C₃₋₇ cycloalkyl” refers to a cyclic alkyl group having 3 to 7 carbon atoms, such as a cyclopropyl, cyclobutyl, cyclopentyl, cyclohexyl, cycloheptyl, or similar groups.

The term “cycloalkenyl” as used herein has the meaning commonly understood by a skilled person. For example, the term “C₅₋₇ cycloalkenyl” refers to a cyclic alkenyl group having 5 to 7 carbon atoms and one or more double bonds, such as a cyclopentenyl, cyclohexenyl, cycloheptenyl, 1,3-cyclohexadienyl, 1,4-cyclohexadienyl, or similar groups.

The term “alkoxy” as used herein has the meaning commonly understood by a skilled person. For example, the term “C₁₋₄ alkoxy” refers to a straight-chain or branched alkoxy group having 1 to 4 carbon atoms, such as a methoxy, ethoxy, propoxy, isopropoxy, butoxy, isobutoxy, sec-butoxy, tert-butoxy, or similar groups.

The term “halogen” as used herein has the meaning commonly understood by a skilled person. For example, the term “halogen” refers to a fluorine, chlorine, bromine, or iodine. The term “halo” refers to a group substituted with one or more of the aforementioned halogen atoms, which may be the same or different, such as a trifluoromethyl, pentafluoroethyl, heptafluoroisopropyl, or similar groups.

The term “ring” refers to a carbocycle or a heterocycle.

The term “heterocycle” refers to a ring in which at least one atom forming the ring skeleton is not a carbon, but is a nitrogen, oxygen, or sulfur. Typically, a heterocycle contains no more than four nitrogen atoms, no more than two oxygen atoms, and/or no more than two sulfur atoms. Unless otherwise specified, the heterocycle may be saturated, partially unsaturated, or fully unsaturated.

The term “ring system” refers to a fused ring consisting of two or more rings joined together.

As used herein, the term “5- or 6-membered heterocyclyl” refers to a five- or six-membered ring containing one or more heteroatoms selected from a nitrogen, oxygen, or sulfur, such as pyridyl, thiazolyl, isothiazolyl, thienyl, furyl, pyrrolyl, pyrazolyl, pyrimidinyl, tetrahydrofuryl, 4,5-dihydrothiazol-2-yl, 2-cyanoimino-4-oxo-1,3-thiazolidin-3-yl, 2-cyanoimino-4-oxo-1,3-thiazinan-3-yl, oxazolyl, isoxazolyl, 1H-tetrazolyl, 1H-1,2,3-triazolyl, 4H-1,2,4-triazolyl, 1,2,3-thiadiazolyl, 1,2,4-thiadiazolyl, 1,3,4-thiadiazolyl, 1,2,4-oxadiazolyl, 1,2,5-oxadiazolyl, 1,3,4-oxadiazolyl, or tetrazolyl, and the like.

The term “heterocyclic ring system” refers to a ring system in which at least one ring is a heterocycle.

The term “heteroaromatic ring system” refers to a ring system in which at least one ring is an aromatic ring.

As used herein, the term “8- to 12-membered heteroaryl bicyclic ring system” or “8- to 14-membered heteroaryl bicyclic or tricyclic ring system” may be selected from the group consisting of: benzofuran, benzo[b]thiophene, indole, quinoline, isoquinoline, 1H-indazole, 1H-benzo[d]imidazole, benzo[d]thiazole, benzo[d]oxazole, benzo[d]isoxazole, benzo[d][1,2,3]thiadiazole, 2,3-dihydroimidazo[1,2-a]pyridine, quinazoline, quinoxaline, cinnoline, phthalazine, 1,8-naphthyridine, 4,5,6,7-tetrahydrobenzo[b]thiophene, benzo[b]thiophene-1,1-dioxide, 8H-indeno[2,1-b]thiophene, 7,8-dihydro-6H-cyclopenta[4,5]thieno[2,3-d]pyrimidine, 3,5,6,7-tetrahydro-4H-cyclopenta[4,5]thieno[2,3-d]pyrimidin-4-one, spiro[indoline-3,2′-[1,3]dioxolane]-2-one, spiro[indoline-3,2′-[1,3]dioxane]-2-one, or indolin-2,3-dione, and the like.

Unless otherwise specified, the groups of the present invention may be substituted by a substituent selected from the group consisting of: a halogen, cyano, nitro, C₁₋₆ alkyl, C₁₋₆ haloalkyl, C₂₋₆ alkenyl, C₂₋₆ haloalkenyl, C₂₋₆ alkynyl, C₂₋₆ haloalkynyl, hydroxy, hydroxy-C₁₋₄ alkyl, OR³, NR³R⁴, C(O)R³, C(O)OR³, C(O)NR³R⁴, SR³, S(O)ₘR⁵, S(O)₂NR³R⁴, OC(O)R⁵, OC(O)NR³R⁴, OS(O)₂R⁵, OS(O)₂NR³R⁴, N(R⁶)C(O)R⁵, N(R⁶)C(O)NR³R⁴, N(R⁶)S(O)₂R⁵ or N(R⁶)S(O)₂NR³R⁴, and the like, wherein R³, R⁴, R⁵, and R⁶ are as defined above, and m is 1 or 2.

The term “substituted” as used herein refers to the replacement of one or more hydrogen atoms on a specific group with a particular substituent. The particular substituent may be the same as the substituents as described above, or it may be a specific substituent appearing in the various examples. Therefore, in the present invention, the substituents in the general formula may each independently be the corresponding groups in the specific compounds of the examples; that is, the present invention includes combinations of the various substituents in the aforementioned general formula, as well as combinations of some of the substituents shown in the general formula with other specific substituents appearing in the examples, provided that the intended combination of substituents is stable or chemically feasible.

Unless otherwise specified, a substituted group may have a specific substituent at any substitutable position of that group, and the said substituent may be the same or different at each position.

An inert solvent refers to various solvents that do not react with the starting materials, including straight-chain, branched, or cyclic alcohols, ethers, or ketones, halogenated alkanes, 1,4-dioxane, acetonitrile, tetrahydrofuran, N,N-dimethylformamide (DMF), dimethyl sulfoxide (DMSO), and the like.

The compounds of the present invention may contain one or more asymmetric centers and thus may exist as racemates, racemic mixtures, single enantiomers, diastereoisomeric compounds, and single diastereoisomers. The possible asymmetric centers depend on the nature of the various substituents of the molecule. Each such asymmetric center will independently produce two optical isomers, and all possible optical isomers, diastereoisomeric mixtures, and pure or partially pure compounds are included within the scope of the present invention. The invention encompasses all isomeric forms of the compounds.

### Compounds of the Present Invention

As used herein, terms “the compound of the present invention” and “the compound shown in Formula I” have the same meaning, both referring to a compound having the structure shown in Formula I.

The compound of Formula I of the present invention exhibits excellent synergistic effects for antibiotics against Gram-negative bacteria or polypeptide antibiotics, therefore, the compound of Formula I of the present invention, as well as various crystal forms, pharmaceutically acceptable inorganic or organic salts, hydrates or solvates thereof, and a pharmaceutical composition comprising the compound of Formula I and an antibiotic as the main active ingredients, can be used for treating, preventing and alleviating diseases related to bacterial infections.

In a preferred embodiment of the present invention, structures of representative compounds are shown below in Table A.

| Number | 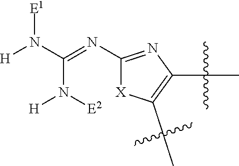 | G | E³ |
|---|---|---|---|
| I-1 | 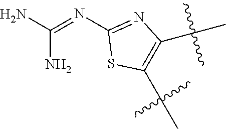 [SMILES: *c1nc(N=C(N)N)sc1*] | 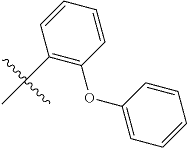 [SMILES: *c1ccccc1Oc1ccccc1] | H |
| I-2 | 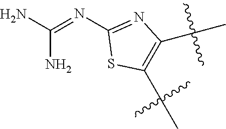 [SMILES: *c1nc(N=C(N)N)sc1*] | 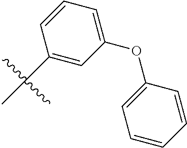 [SMILES: *c1cccc(Oc2ccccc2)c1] | H |
| I-3 | 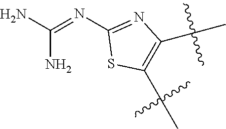 [SMILES: *c1nc(N=C(N)N)sc1*] | 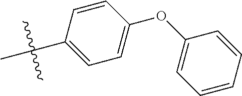 [SMILES: *c1ccc(Oc2ccccc2)cc1] | H |
| I-4 | 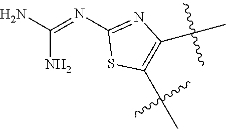 [SMILES: *c1nc(N=C(N)N)sc1*] | 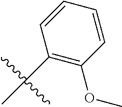 [SMILES: *c1ccccc1OC] | H |
| I-5 | 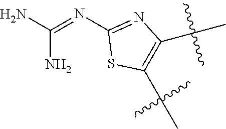 [SMILES: *c1nc(N=C(N)N)sc1*] | 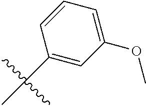 [SMILES: *c1cccc(OC)c1] | H |
| I-6 | 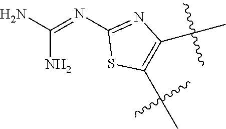 [SMILES: *c1nc(N=C(N)N)sc1*] | 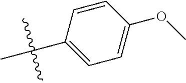 [SMILES: *c1ccc(OC)cc1] | H |
| I-7 | 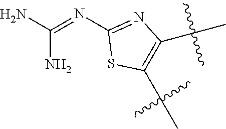 [SMILES: *c1nc(N=C(N)N)sc1*] | 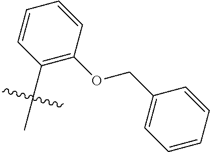 [SMILES: *c1ccccc1OCc1ccccc1] | H |
| I-8 | 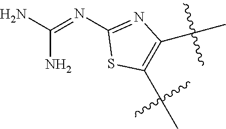 [SMILES: *c1nc(N=C(N)N)sc1*] | 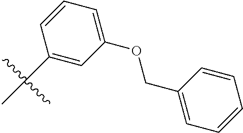 [SMILES: *c1cccc(OCc2ccccc2)c1] | H |
| I-9 | 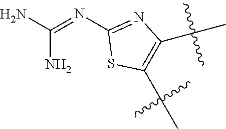 [SMILES: *c1nc(N=C(N)N)sc1*] | 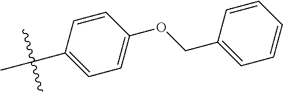 [SMILES: *c1ccc(OCc2ccccc2)cc1] | H |
| I-10 | 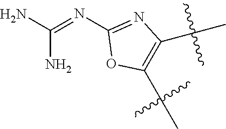 [SMILES: *c1nc(N=C(N)N)oc1*] | 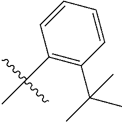 [SMILES: *c1ccccc1C(C)(C)C] | H |
| I-11 | 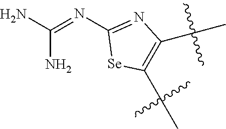 [SMILES: *c1nc(N=C(N)N)[se]c1*] | 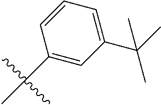 [SMILES: *c1cccc(C(C)(C)C)c1] | H |
| I-12 | 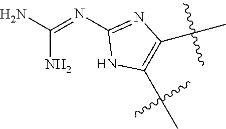 [SMILES: *c1nc(N=C(N)N)[nH]c1*] | 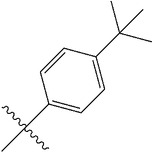 [SMILES: *c1ccc(C(C)(C)C)cc1] | H |
| I-13 | 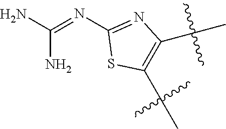 [SMILES: *c1nc(N=C(N)N)sc1*] | 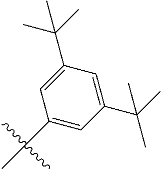 [SMILES: *c1cc(C(C)(C)C)cc(C(C)(C)C)c1] | H |
| I-14 | 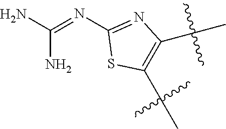 [SMILES: *c1nc(N=C(N)N)sc1*] | 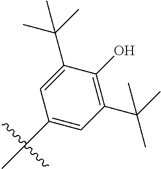 [SMILES: *c1cc(C(C)(C)C)c(O)c(C(C)(C)C)c1] | H |
| I-15 | 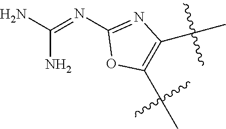 [SMILES: *c1nc(N=C(N)N)oc1*] | 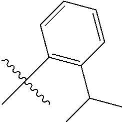 [SMILES: *c1ccccc1C(C)C] | H |
| I-16 | 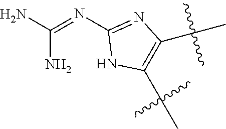 [SMILES: *c1nc(N=C(N)N)[nH]c1*] | 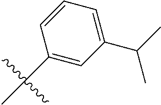 [SMILES: *c1cccc(C(C)C)c1] | H |
| I-17 | 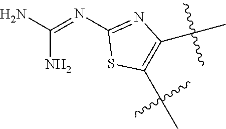 [SMILES: *c1nc(N=C(N)N)sc1*] | 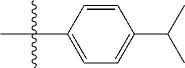 [SMILES: *c1ccc(C(C)C)cc1] | H |
| I-18 | 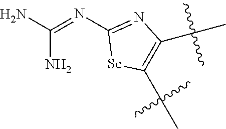 [SMILES: *c1nc(N=C(N)N)[se]c1*] | 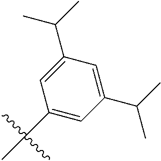 [SMILES: *c1cc(C(C)C)cc(C(C)C)c1] | H |
| I-19 | 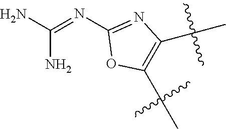 [SMILES: *c1nc(N=C(N)N)oc1*] | 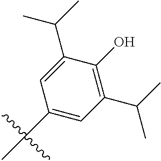 [SMILES: *c1cc(C(C)C)c(O)c(C(C)C)c1] | H |
| I-20 | 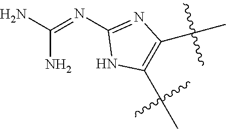 [SMILES: *c1nc(N=C(N)N)[nH]c1*] | 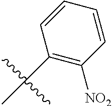 [SMILES: *c1ccccc1[N+](=O)[O-]] | H |
| I-21 | 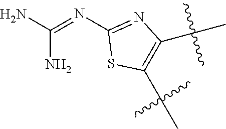 [SMILES: *c1nc(N=C(N)N)sc1*] | 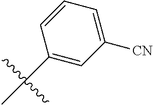 [SMILES: *c1cccc(C#N)c1] | H |
| I-22 | 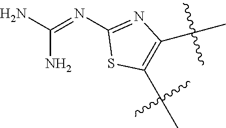 [SMILES: *c1nc(N=C(N)N)sc1*] | 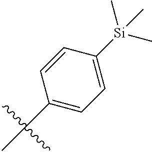 [SMILES: *c1ccc([Si](C)(C)C)cc1] | H |
| I-23 | 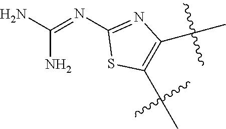 [SMILES: *c1nc(N=C(N)N)sc1*] | 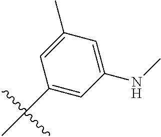 [SMILES: *c1cc(C)cc(NC)c1] | H |
| I-24 | 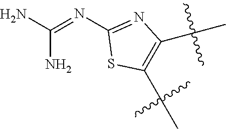 [SMILES: *c1nc(N=C(N)N)sc1*] | 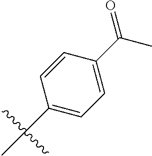 [SMILES: *c1ccc(C(C)=O)cc1] | H |
| I-25 | 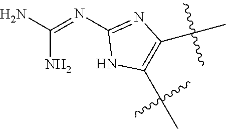 [SMILES: *c1nc(N=C(N)N)[nH]c1*] | 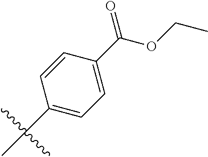 [SMILES: *c1ccc(C(=O)OCC)cc1] | H |
| I-26 | 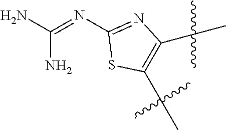 [SMILES: *c1nc(N=C(N)N)sc1*] | 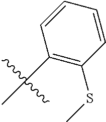 [SMILES: *c1ccccc1SC] | H |
| I-27 | 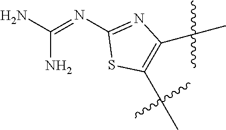 [SMILES: *c1nc(N=C(N)N)sc1*] | 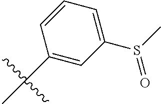 [SMILES: *c1cccc(S(C)=O)c1] | H |
| I-28 | 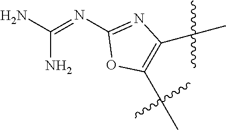 [SMILES: *c1nc(N=C(N)N)oc1*] | 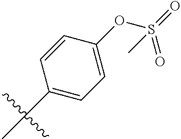 [SMILES: *c1ccc(OS(C)(=O)=O)cc1] | H |
| I-29 | 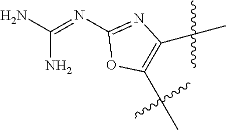 [SMILES: *c1nc(N=C(N)N)oc1*] | 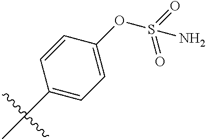 [SMILES: *c1ccc(OS(N)(=O)=O)cc1] | H |
| I-30 | 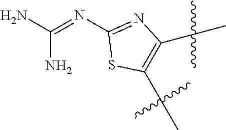 [SMILES: *c1nc(N=C(N)N)sc1*] | 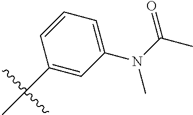 [SMILES: *c1cccc(N(C)C(C)=O)c1] | H |
| I-31 | 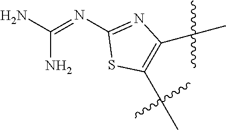 [SMILES: *c1nc(N=C(N)N)sc1*] | 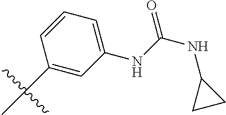 [SMILES: *c1cccc(NC(=O)NC2CC2)c1] | H |
| I-32 | 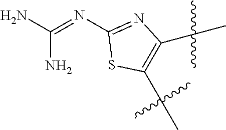 [SMILES: *c1nc(N=C(N)N)sc1*] | 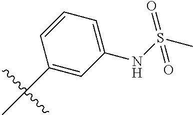 [SMILES: *c1cccc(NS(C)(=O)=O)c1] | H |
| I-33 | 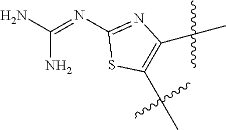 [SMILES: *c1nc(N=C(N)N)sc1*] | 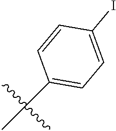 [SMILES: *c1ccc(I)cc1] | H |
| I-34 | 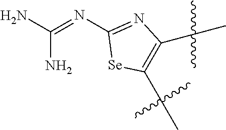 [SMILES: *c1nc(N=C(N)N)[se]c1*] | 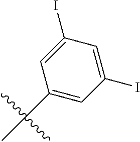 [SMILES: *c1cc(I)cc(I)c1] | H |
| I-35 | 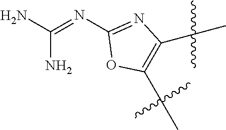 [SMILES: *c1nc(N=C(N)N)oc1*] | 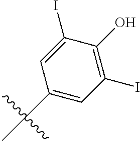 [SMILES: *c1cc(I)c(O)c(I)c1] | H |
| I-36 | 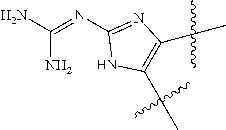 [SMILES: *c1nc(N=C(N)N)[nH]c1*] | 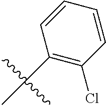 [SMILES: *c1ccccc1Cl] | H |
| I-37 | 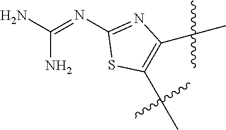 [SMILES: *c1nc(N=C(N)N)sc1*] | 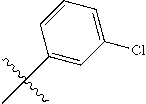 [SMILES: *c1cccc(Cl)c1] | H |
| I-38 | 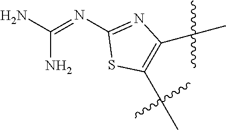 [SMILES: *c1nc(N=C(N)N)sc1*] | 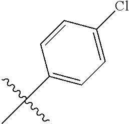 [SMILES: *c1ccc(Cl)cc1] | H |
| I-39 | 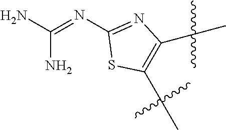 [SMILES: *c1nc(N=C(N)N)sc1*] | 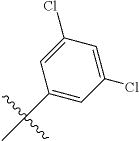 [SMILES: *c1cc(Cl)cc(Cl)c1] | H |
| I-40 | 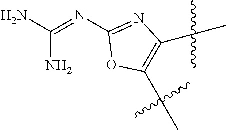 [SMILES: *c1nc(N=C(N)N)oc1*] | 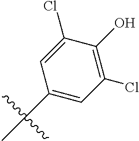 [SMILES: *c1cc(Cl)c(O)c(Cl)c1] | H |
| I-41 | 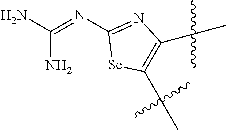 [SMILES: *c1nc(N=C(N)N)[se]c1*] | 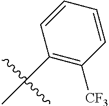 [SMILES: *c1ccccc1C(F)(F)F] | H |
| I-42 | 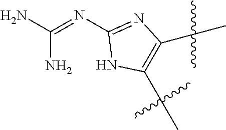 [SMILES: *c1nc(N=C(N)N)[nH]c1*] | 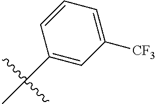 [SMILES: *c1cccc(C(F)(F)F)c1] | H |
| I-43 | 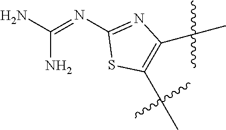 [SMILES: *c1nc(N=C(N)N)sc1*] | 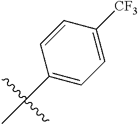 [SMILES: *c1ccc(C(F)(F)F)cc1] | H |
| I-44 | 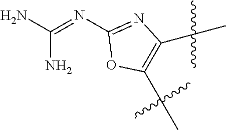 [SMILES: *c1nc(N=C(N)N)oc1*] | 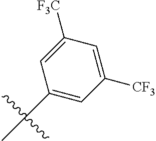 [SMILES: *c1cc(C(F)(F)F)cc(C(F)(F)F)c1] | H |
| I-45 | 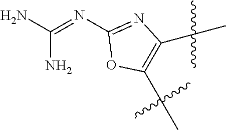 [SMILES: *c1nc(N=C(N)N)oc1*] | 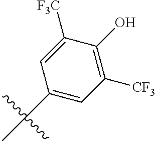 [SMILES: *c1cc(C(F)(F)F)c(O)c(C(F)(F)F)c1] | H |
| I-46 | 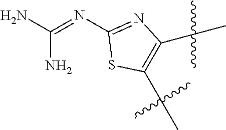 [SMILES: *c1nc(N=C(N)N)sc1*] | 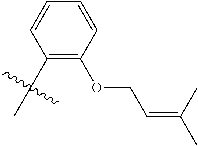 [SMILES: *c1ccccc1OCC=C(C)C] | H |
| I-47 | 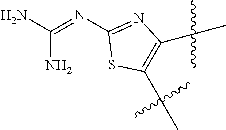 [SMILES: *c1nc(N=C(N)N)sc1*] | 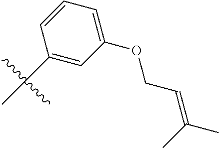 [SMILES: *c1cccc(OCC=C(C)C)c1] | H |
| I-48 | 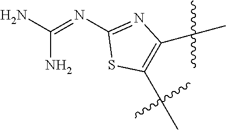 [SMILES: *c1nc(N=C(N)N)sc1*] | 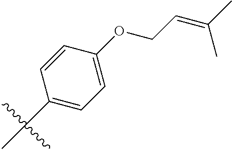 [SMILES: *c1ccc(OCC=C(C)C)cc1] | H |
| I-49 | 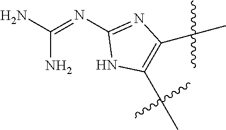 [SMILES: *c1nc(N=C(N)N)[nH]c1*] | 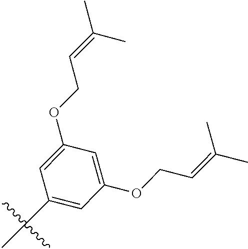 [SMILES: *c1cc(OCC=C(C)C)cc(OCC=C(C)C)c1] | Me |
| I-50 | 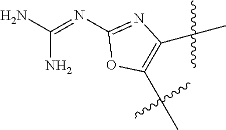 [SMILES: *c1nc(N=C(N)N)oc1*] | 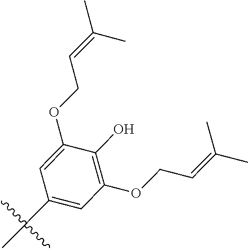 [SMILES: *c1cc(OCC=C(C)C)c(O)c(OCC=C(C)C)c1] | Cl |
| I-51 | 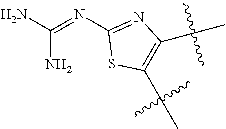 [SMILES: *c1nc(N=C(N)N)sc1*] | 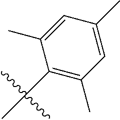 | H |
| I-52 | 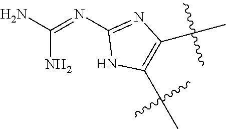 [SMILES: *c1nc(N=C(N)N)[nH]c1*] | 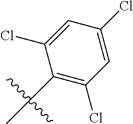 [SMILES: *c1c(Cl)cc(Cl)cc1Cl] | H |
| I-53 | 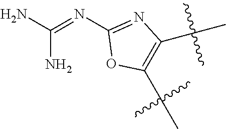 [SMILES: *c1nc(N=C(N)N)oc1*] | 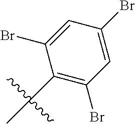 [SMILES: *c1c(Br)cc(Br)cc1Br] | I |
| I-54 | 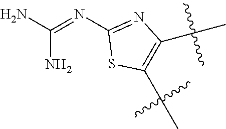 [SMILES: *c1nc(N=C(N)N)sc1*] | 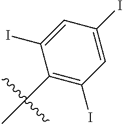 [SMILES: *c1c(I)cc(I)cc1I] | H |
| I-55 | 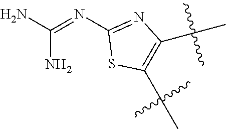 [SMILES: *c1nc(N=C(N)N)sc1*] | 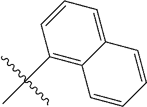 | H |
| I-56 | 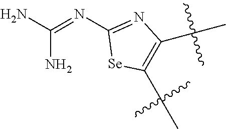 [SMILES: *c1nc(N=C(N)N)[se]c1*] | 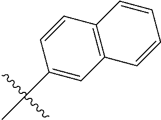 [SMILES: *c1ccc2ccccc2c1] | H |
| I-57 | 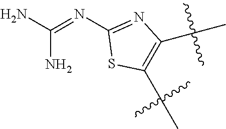 [SMILES: *c1nc(N=C(N)N)sc1*] | 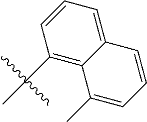 | H |
| I-58 | 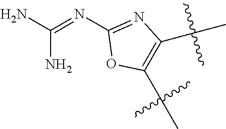 [SMILES: *c1nc(N=C(N)N)oc1*] | 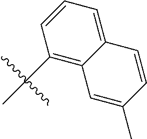 | Me |
| I-59 | 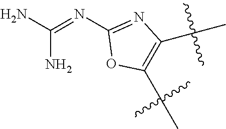 [SMILES: *c1nc(N=C(N)N)oc1*] | 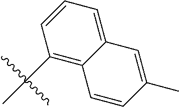 | H |
| I-60 | 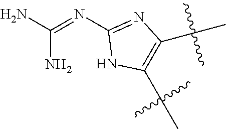 [SMILES: *c1nc(N=C(N)N)[nH]c1*] | 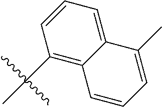 | Et |
| I-61 | 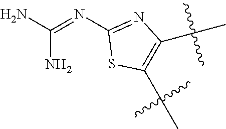 [SMILES: *c1nc(N=C(N)N)sc1*] | 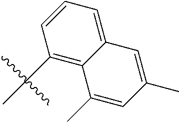 | H |
| I-62 | 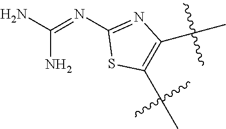 [SMILES: *c1nc(N=C(N)N)sc1*] | 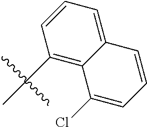 | Cl |
| I-63 | 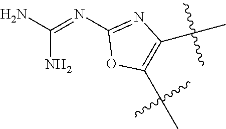 [SMILES: *c1nc(N=C(N)N)oc1*] | 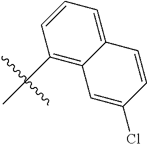 | Me |
| I-64 | 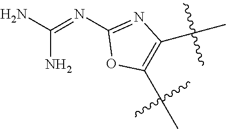 [SMILES: *c1nc(N=C(N)N)oc1*] | 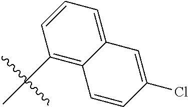 | H |
| I-65 | 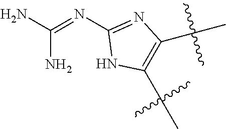 [SMILES: *c1nc(N=C(N)N)[nH]c1*] | 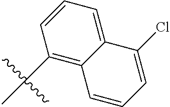 [SMILES: *c1cccc2c(Cl)cccc12] | H |
| I-66 | 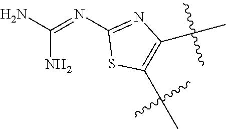 [SMILES: *c1nc(N=C(N)N)sc1*] | 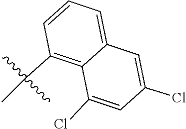 [SMILES: *c1cccc2cc(Cl)cc(Cl)c12] | Br |
| I-67 | 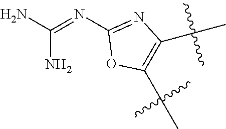 [SMILES: *c1nc(N=C(N)N)oc1*] | 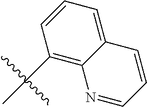 | H |
| I-68 | 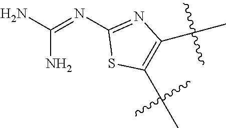 [SMILES: *c1nc(N=C(N)N)sc1*] | 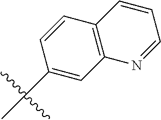 [SMILES: *c1ccc2cccnc2c1] | H |
| I-69 | 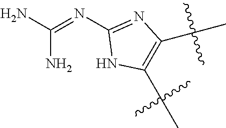 [SMILES: *c1nc(N=C(N)N)[nH]c1*] | 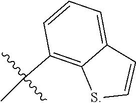 [SMILES: *c1cccc2ccsc12] | H |
| I-70 | 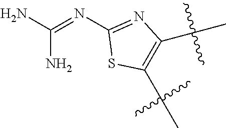 [SMILES: *c1nc(N=C(N)N)sc1*] | 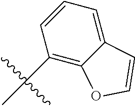 [SMILES: *c1cccc2ccoc12] | Me |
| I-71 | 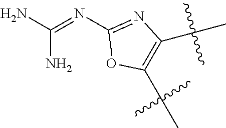 [SMILES: *c1nc(N=C(N)N)oc1*] | 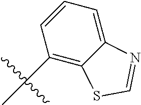 [SMILES: *c1cccc2ncsc12] | H |
| I-72 | 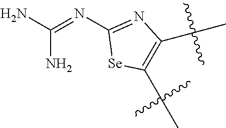 [SMILES: *c1nc(N=C(N)N)[se]c1*] | 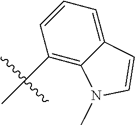 [SMILES: *c1cccc2ccn(C)c12] | H |
| I-73 | 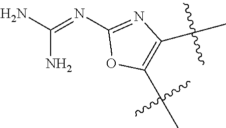 [SMILES: *c1nc(N=C(N)N)oc1*] | 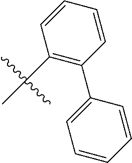 [SMILES: *c1ccccc1-c1ccccc1] | H |
| I-74 | 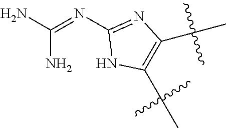 [SMILES: *c1nc(N=C(N)N)[nH]c1*] | 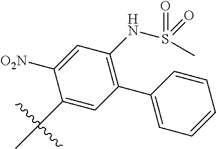 [SMILES: *c1cc(-c2ccccc2)c(NS(C)(=O)=O)cc1[N+](=O)[O-]] | H |
| I-75 | 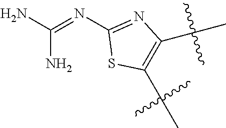 [SMILES: *c1nc(N=C(N)N)sc1*] | 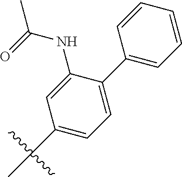 [SMILES: *c1ccc(-c2ccccc2)c(NC(C)=O)c1] | H |
| I-76 | 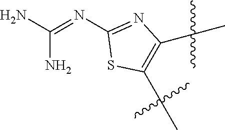 [SMILES: *c1nc(N=C(N)N)sc1*] | 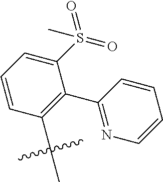 [SMILES: *c1cccc(S(C)(=O)=O)c1-c1ccccn1] | Me |
| I-77 | 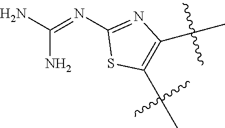 [SMILES: *c1nc(N=C(N)N)sc1*] | 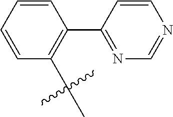 [SMILES: *c1ccccc1-c1ccncn1] | Et |
| I-78 | 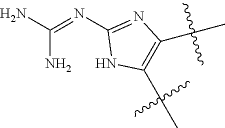 [SMILES: *c1nc(N=C(N)N)[nH]c1*] | 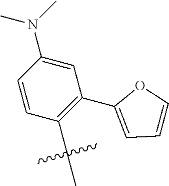 [SMILES: *c1ccc(N(C)C)cc1-c1ccco1] | Cl |
| I-79 | 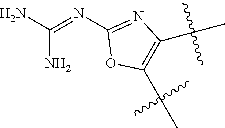 [SMILES: *c1nc(N=C(N)N)oc1*] | 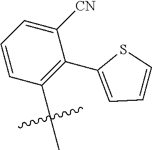 [SMILES: *c1cccc(C#N)c1-c1cccs1] | Me |
| I-80 | 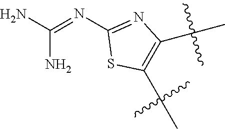 [SMILES: *c1nc(N=C(N)N)sc1*] | 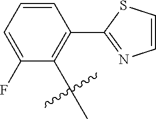 [SMILES: *c1c(F)cccc1-c1nccs1] | H |
| I-81 | 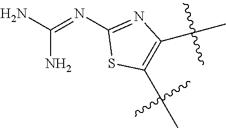 [SMILES: *c1nc(N=C(N)N)sc1*] | 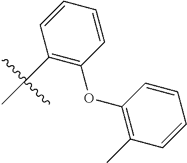 [SMILES: *c1ccccc1Oc1ccccc1C] | H |
| I-82 | 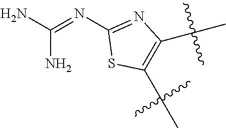 [SMILES: *c1nc(N=C(N)N)sc1*] | 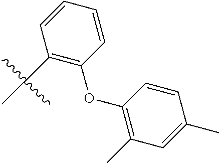 [SMILES: *c1ccccc1Oc1ccc(C)cc1C] | H |
| I-83 | 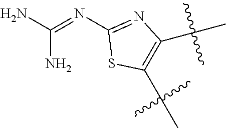 [SMILES: *c1nc(N=C(N)N)sc1*] | 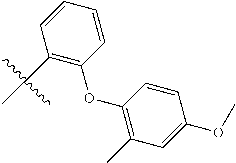 [SMILES: *c1ccccc1Oc1ccc(OC)cc1C] | H |
| I-84 | 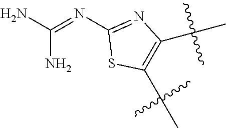 [SMILES: *c1nc(N=C(N)N)sc1*] | 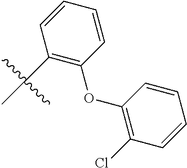 [SMILES: *c1ccccc1Oc1ccccc1Cl] | H |
| I-85 | 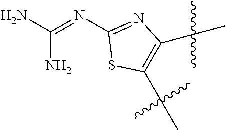 [SMILES: *c1nc(N=C(N)N)sc1*] | 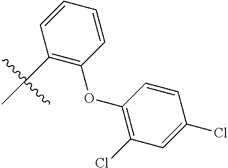 [SMILES: *c1ccccc1Oc1ccc(Cl)cc1Cl] | H |
| I-86 | 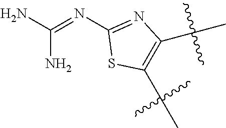 [SMILES: *c1nc(N=C(N)N)sc1*] | 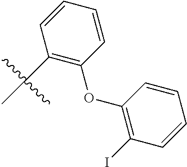 [SMILES: *c1ccccc1Oc1ccccc1I] | H |
| I-87 | 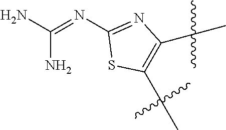 [SMILES: *c1nc(N=C(N)N)sc1*] | 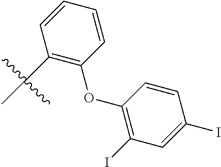 [SMILES: *c1ccccc1Oc1ccc(I)cc1I] | H |
| I-88 | 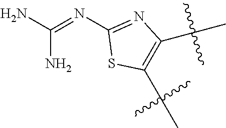 [SMILES: *c1nc(N=C(N)N)sc1*] | 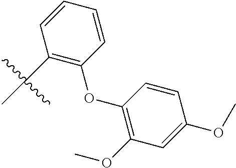 [SMILES: *c1ccccc1Oc1ccc(OC)cc1OC] | H |
| I-89 | 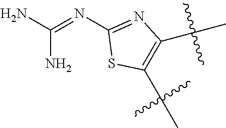 [SMILES: *c1nc(N=C(N)N)sc1*] | 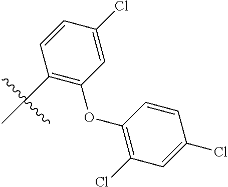 [SMILES: *c1ccc(Cl)cc1Oc1ccc(Cl)cc1Cl] | Me |
| I-90 | 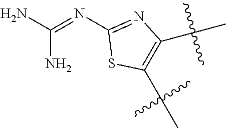 [SMILES: *c1nc(N=C(N)N)sc1*] | 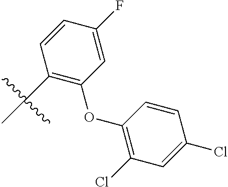 [SMILES: *c1ccc(F)cc1Oc1ccc(Cl)cc1Cl] | Cl |
| I-91 | 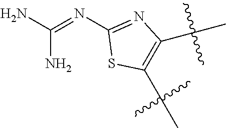 [SMILES: *c1nc(N=C(N)N)sc1*] | 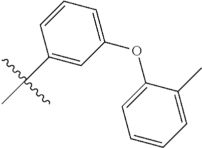 [SMILES: *c1cccc(Oc2ccccc2C)c1] | H |
| I-92 | 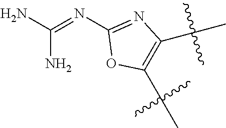 [SMILES: *c1nc(N=C(N)N)oc1*] | 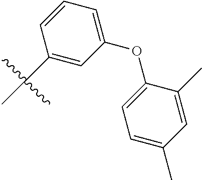 [SMILES: *c1cccc(Oc2ccc(C)cc2C)c1] | H |
| I-93 | 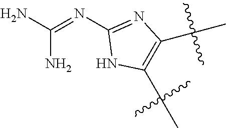 [SMILES: *c1nc(N=C(N)N)[nH]c1*] | 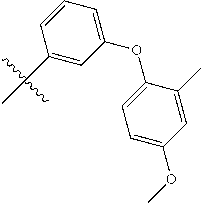 [SMILES: *c1cccc(Oc2ccc(OC)cc2C)c1] | H |
| I-94 | 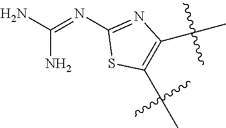 [SMILES: *c1nc(N=C(N)N)sc1*] | 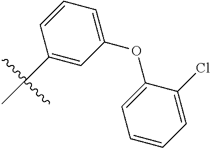 [SMILES: *c1cccc(Oc2ccccc2Cl)c1] | H |
| I-95 | 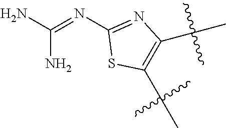 [SMILES: *c1nc(N=C(N)N)sc1*] | 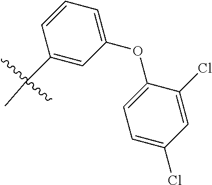 [SMILES: *c1cccc(Oc2ccc(Cl)cc2Cl)c1] | H |
| I-96 | 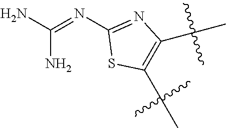 [SMILES: *c1nc(N=C(N)N)sc1*] | 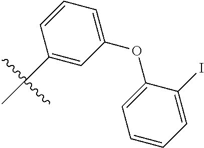 [SMILES: *c1cccc(Oc2ccccc2I)c1] | H |
| I-97 | 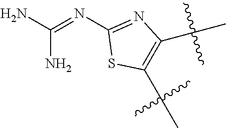 [SMILES: *c1nc(N=C(N)N)sc1*] | 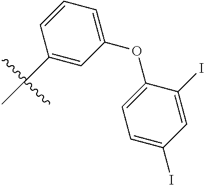 [SMILES: *c1cccc(Oc2ccc(I)cc2I)c1] | H |
| I-98 | 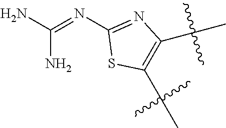 [SMILES: *c1nc(N=C(N)N)sc1*] | 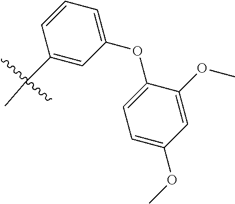 [SMILES: *c1cccc(Oc2ccc(OC)cc2OC)c1] | H |
| I-99 | 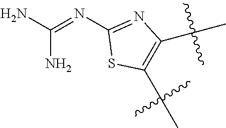 [SMILES: *c1nc(N=C(N)N)sc1*] | 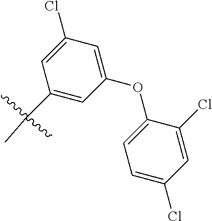 [SMILES: *c1cc(Cl)cc(Oc2ccc(Cl)cc2Cl)c1] | H |
| I-100 | 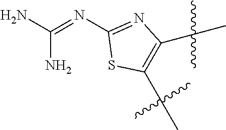 [SMILES: *c1nc(N=C(N)N)sc1*] | 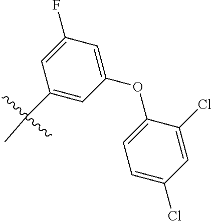 [SMILES: *c1cc(F)cc(Oc2ccc(Cl)cc2Cl)c1] | H |
| I-101 | 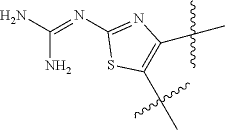 [SMILES: *c1nc(N=C(N)N)sc1*] | 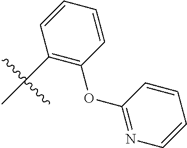 [SMILES: *c1ccccc1Oc1ccccn1] | Me |
| I-102 | 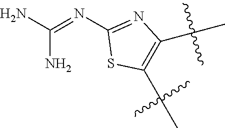 [SMILES: *c1nc(N=C(N)N)sc1*] | 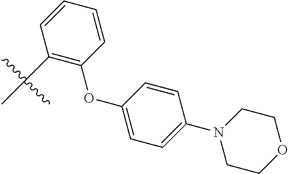 [SMILES: *c1ccccc1Oc1ccc(N2CCOCC2)cc1] | Et |
| I-103 | 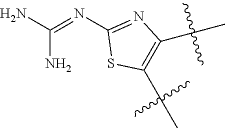 [SMILES: *c1nc(N=C(N)N)sc1*] | 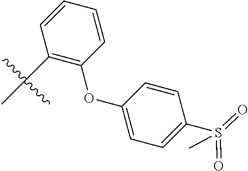 [SMILES: *c1ccccc1Oc1ccc(S(C)(=O)=O)cc1] | Cl |
| I-104 | 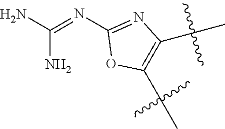 [SMILES: *c1nc(N=C(N)N)oc1*] | 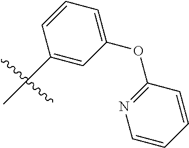 [SMILES: *c1cccc(Oc2ccccn2)c1] | H |
| I-105 | 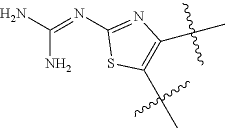 [SMILES: *c1nc(N=C(N)N)sc1*] | 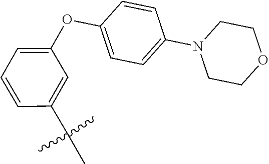 [SMILES: *c1cccc(Oc2ccc(N3CCOCC3)cc2)c1] | H |
| I-106 | 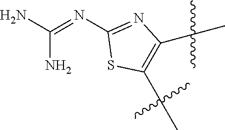 [SMILES: *c1nc(N=C(N)N)sc1*] | 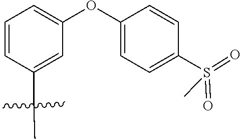 [SMILES: *c1cccc(Oc2ccc(S(C)(=O)=O)cc2)c1] | H |
| I-107 | 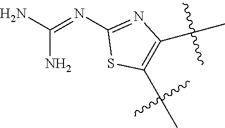 [SMILES: *c1nc(N=C(N)N)sc1*] | 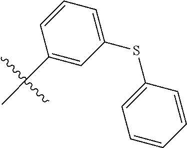 [SMILES: *c1cccc(Sc2ccccc2)c1] | H |
| I-108 | 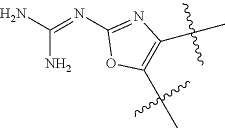 [SMILES: *c1nc(N=C(N)N)oc1*] | 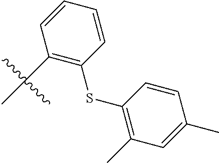 [SMILES: *c1ccccc1Sc1ccc(C)cc1C] | H |
| I-109 | 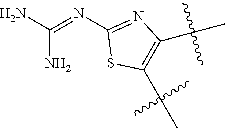 [SMILES: *c1nc(N=C(N)N)sc1*] | 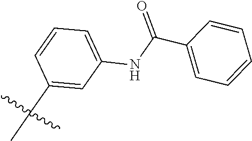 [SMILES: *c1cccc(NC(=O)c2ccccc2)c1] | H |
| I-110 | 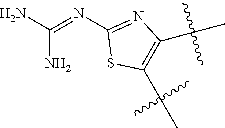 [SMILES: *c1nc(N=C(N)N)sc1*] | 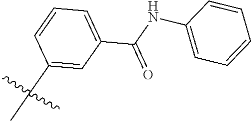 [SMILES: *c1cccc(C(=O)Nc2ccccc2)c1] | H |
| I-111 | 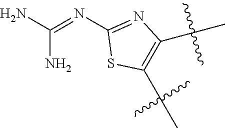 [SMILES: *c1nc(N=C(N)N)sc1*] | 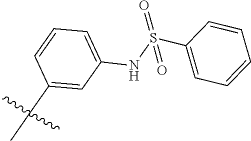 [SMILES: *c1cccc(NS(=O)(=O)c2ccccc2)c1] | H |
| I-112 | 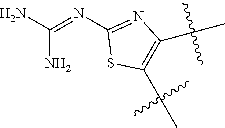 [SMILES: *c1nc(N=C(N)N)sc1*] | 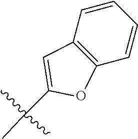 [SMILES: *c1cc2ccccc2o1] | H |
| I-113 | 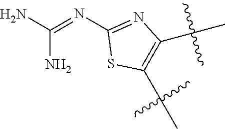 [SMILES: *c1nc(N=C(N)N)sc1*] | 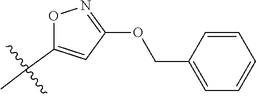 [SMILES: *c1cc(OCc2ccccc2)no1] | H |
| I-114 | 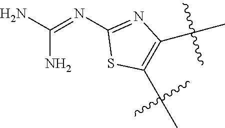 [SMILES: *c1nc(N=C(N)N)sc1*] | 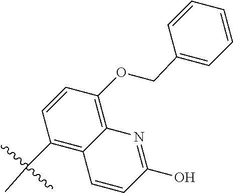 [SMILES: *c1ccc(OCc2ccccc2)c2nc(O)ccc12] | H |
| I-115 | 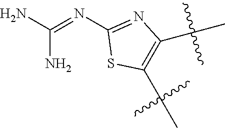 [SMILES: *c1nc(N=C(N)N)sc1*] | 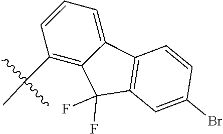 [SMILES: *c1cccc2c1C(F)(F)c1cc(Br)ccc1-2] | H |
| I-116 | 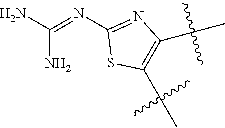 [SMILES: *c1nc(N=C(N)N)sc1*] | 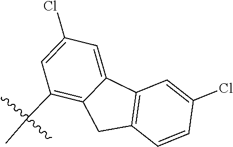 [SMILES: *c1cc(Cl)cc2c1Cc1ccc(Cl)cc1-2] | H |
| I-117 | 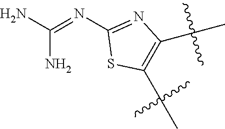 [SMILES: *c1nc(N=C(N)N)sc1*] | 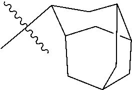 [SMILES: *C1CC2CC3CC1CC2C3] | H |
| I-118 | 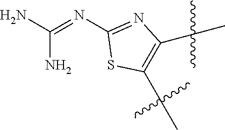 [SMILES: *c1nc(N=C(N)N)sc1*] | 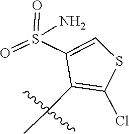 [SMILES: *c1c(S(N)(=O)=O)csc1Cl] | H |
| I-119 | 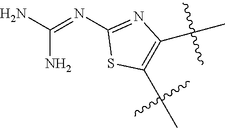 [SMILES: *c1nc(N=C(N)N)sc1*] | 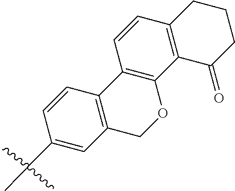 [SMILES: *c1ccc2c(c1)COc1c-2ccc2c1C(=O)CCC2] | H |
| I-120 | 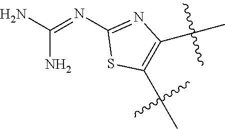 [SMILES: *c1nc(N=C(N)N)sc1*] | 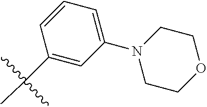 [SMILES: *c1cccc(N2CCOCC2)c1] | H |
| I-121 | 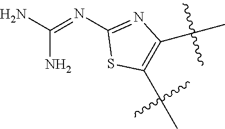 [SMILES: *c1nc(N=C(N)N)sc1*] | 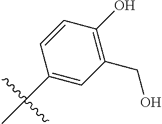 [SMILES: *c1ccc(O)c(CO)c1] | H |
| I-122 | 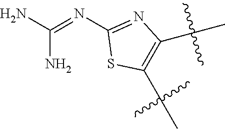 [SMILES: *c1nc(N=C(N)N)sc1*] | 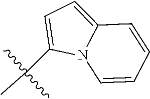 [SMILES: *c1ccc2ccccn12] | H |
| I-123 | 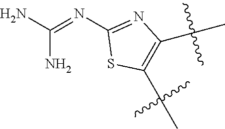 [SMILES: *c1nc(N=C(N)N)sc1*] | 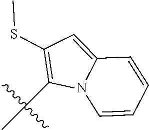 [SMILES: *c1c(SC)cc2ccccn12] | H |
| I-124 | 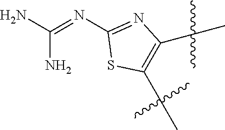 [SMILES: *c1nc(N=C(N)N)sc1*] | 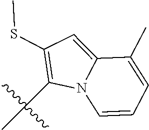 [SMILES: *c1c(SC)cc2c(C)cccn12] | H |
| I-125 | 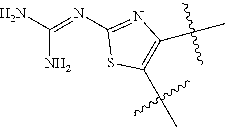 [SMILES: *c1nc(N=C(N)N)sc1*] | 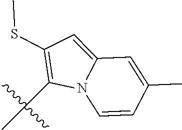 | H |
| I-126 | 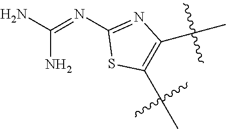 [SMILES: *c1nc(N=C(N)N)sc1*] | 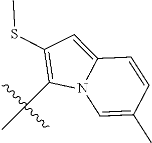 | Me |
| I-127 | 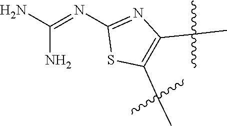 [SMILES: *c1nc(N=C(N)N)sc1*] | 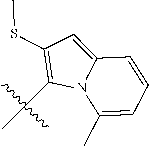 [SMILES: *c1c(SC)cc2cccc(C)n12] | Cl |
| I-128 | 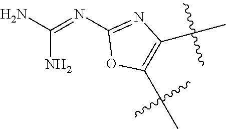 [SMILES: *c1nc(N=C(N)N)oc1*] | 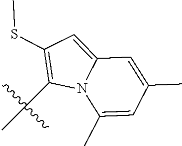 | Me |
| I-129 | 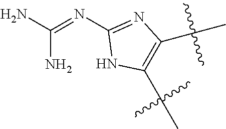 [SMILES: *c1nc(N=C(N)N)[nH]c1*] | 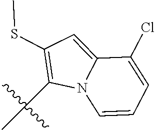 [SMILES: *c1c(SC)cc2c(Cl)cccn12] | H |
| I-130 | 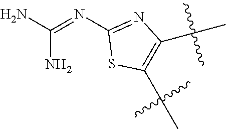 [SMILES: *c1nc(N=C(N)N)sc1*] | 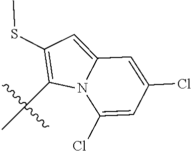 [SMILES: *c1c(SC)cc2cc(Cl)cc(Cl)n12] | Cl |
| I-131 | 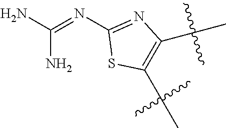 [SMILES: *c1nc(N=C(N)N)sc1*] | 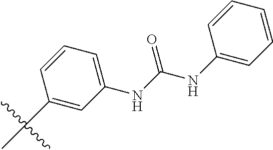 [SMILES: *c1cccc(NC(=O)Nc2ccccc2)c1] | H |
| I-132 | 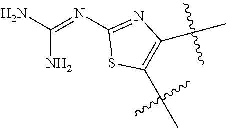 [SMILES: *c1nc(N=C(N)N)sc1*] | 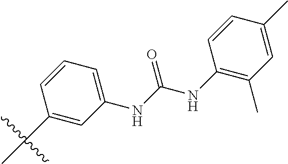 [SMILES: *c1cccc(NC(=O)Nc2ccc(C)cc2C)c1] | Me |
| I-133 | 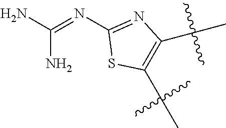 [SMILES: *c1nc(N=C(N)N)sc1*] | 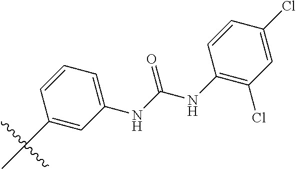 [SMILES: *c1cccc(NC(=O)Nc2ccc(Cl)cc2Cl)c1] | Cl |
| I-134 | 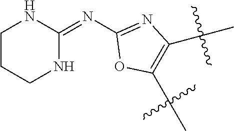 [SMILES: *c1nc(N=C2NCCCN2)oc1*] | 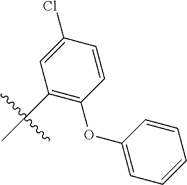 [SMILES: *c1cc(Cl)ccc1Oc1ccccc1] | H |
| I-135 | 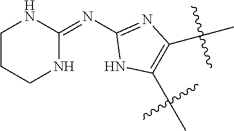 [SMILES: *c1nc(N=C2NCCCN2)[nH]c1*] | 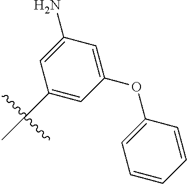 [SMILES: *c1cc(N)cc(Oc2ccccc2)c1] | H |
| I-136 | 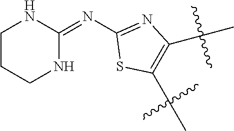 [SMILES: *c1nc(N=C2NCCCN2)sc1*] | 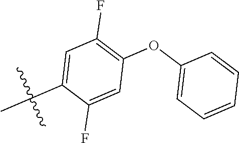 [SMILES: *c1cc(F)c(Oc2ccccc2)cc1F] | H |
| I-137 | 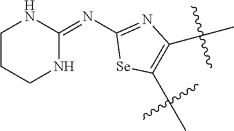 [SMILES: *c1nc(N=C2NCCCN2)[se]c1*] | 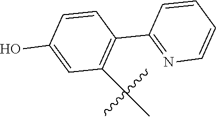 [SMILES: *c1cc(O)ccc1-c1ccccn1] | H |
| I-138 | 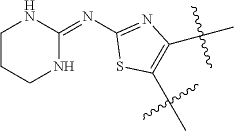 [SMILES: *c1nc(N=C2NCCCN2)sc1*] | 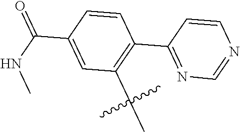 [SMILES: *c1cc(C(=O)NC)ccc1-c1ccncn1] | H |
| I-139 | 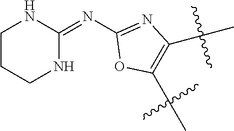 [SMILES: *c1nc(N=C2NCCCN2)oc1*] | 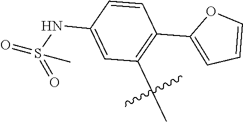 [SMILES: *c1cc(NS(C)(=O)=O)ccc1-c1ccco1] | Me |
| I-140 | 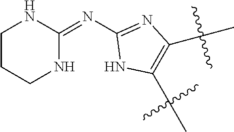 [SMILES: *c1nc(N=C2NCCCN2)[nH]c1*] | 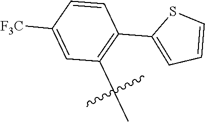 [SMILES: *c1cc(C(F)(F)F)ccc1-c1cccs1] | H |
| I-141 | 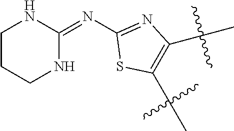 [SMILES: *c1nc(N=C2NCCCN2)sc1*] | 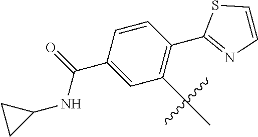 [SMILES: *c1cc(C(=O)NC2CC2)ccc1-c1nccs1] | Br |
| I-142 | 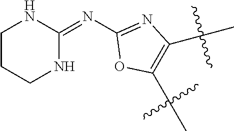 [SMILES: *c1nc(N=C2NCCCN2)oc1*] | 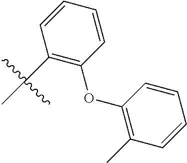 [SMILES: *c1ccccc1Oc1ccccc1C] | Cl |
| I-143 | 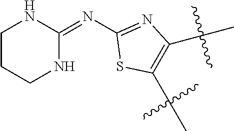 [SMILES: *c1nc(N=C2NCCCN2)sc1*] | 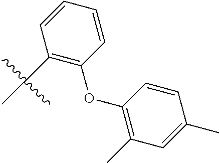 [SMILES: *c1ccccc1Oc1ccc(C)cc1C] | H |
| I-144 | 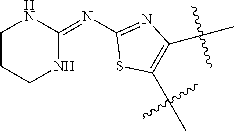 [SMILES: *c1nc(N=C2NCCCN2)sc1*] | 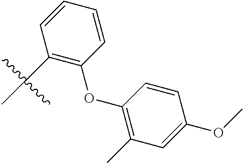 [SMILES: *c1ccccc1Oc1ccc(OC)cc1C] | Me |
| I-145 | 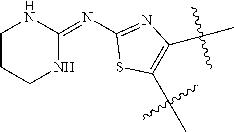 [SMILES: *c1nc(N=C2NCCCN2)sc1*] | 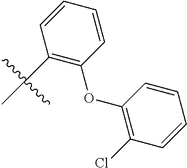 [SMILES: *c1ccccc1Oc1ccccc1Cl] | Me |
| I-146 | 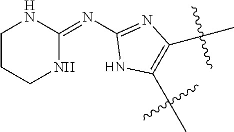 [SMILES: *c1nc(N=C2NCCCN2)[nH]c1*] | 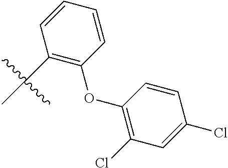 [SMILES: *c1ccccc1Oc1ccc(Cl)cc1Cl] | H |
| I-147 | 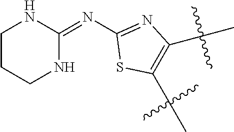 [SMILES: *c1nc(N=C2NCCCN2)sc1*] | 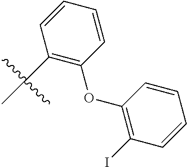 [SMILES: *c1ccccc1Oc1ccccc1I] | H |
| I-148 | 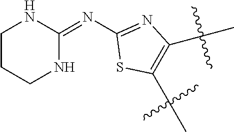 [SMILES: *c1nc(N=C2NCCCN2)sc1*] | 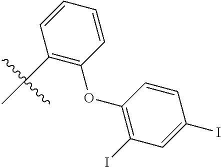 [SMILES: *c1ccccc1Oc1ccc(I)cc1I] | Cl |
| I-149 | 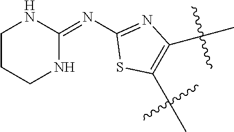 [SMILES: *c1nc(N=C2NCCCN2)sc1*] | 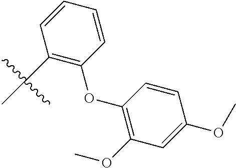 [SMILES: *c1ccccc1Oc1ccc(OC)cc1OC] | H |
| I-150 | 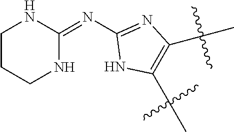 [SMILES: *c1nc(N=C2NCCCN2)[nH]c1*] | 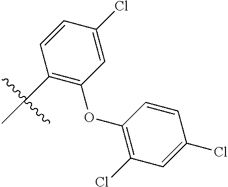 [SMILES: *c1ccc(Cl)cc1Oc1ccc(Cl)cc1Cl] | H |
| I-151 | 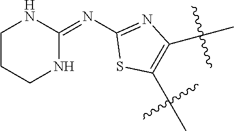 [SMILES: *c1nc(N=C2NCCCN2)sc1*] | 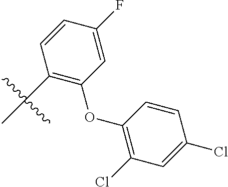 [SMILES: *c1ccc(F)cc1Oc1ccc(Cl)cc1Cl] | H |
| I-152 | 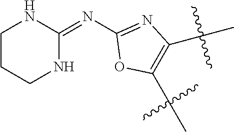 [SMILES: *c1nc(N=C2NCCCN2)oc1*] | 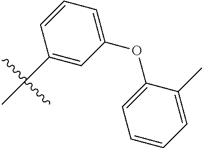 [SMILES: *c1cccc(Oc2ccccc2C)c1] | H |
| I-153 | 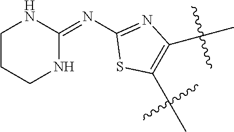 [SMILES: *c1nc(N=C2NCCCN2)sc1*] | 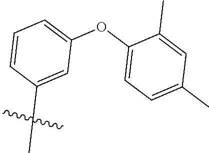 [SMILES: *c1cccc(Oc2ccc(C)cc2C)c1] | H |
| I-154 | 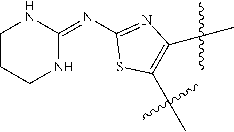 [SMILES: *c1nc(N=C2NCCCN2)sc1*] | 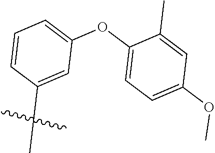 [SMILES: *c1cccc(Oc2ccc(OC)cc2C)c1] | H |
| I-155 | 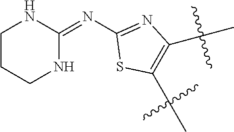 [SMILES: *c1nc(N=C2NCCCN2)sc1*] | 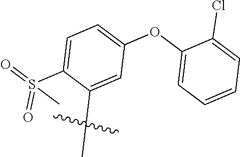 [SMILES: *c1cc(Oc2ccccc2Cl)ccc1S(C)(=O)=O] | Cl |
| I-156 | 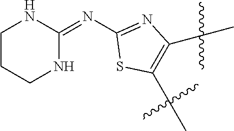 [SMILES: *c1nc(N=C2NCCCN2)sc1*] | 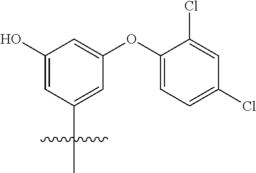 [SMILES: *c1cc(O)cc(Oc2ccc(Cl)cc2Cl)c1] | H |
| I-157 | 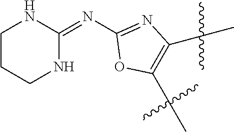 [SMILES: *c1nc(N=C2NCCCN2)oc1*] | 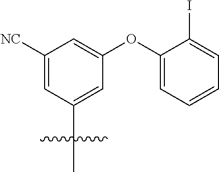 [SMILES: *c1cc(C#N)cc(Oc2ccccc2I)c1] | H |
| I-158 | 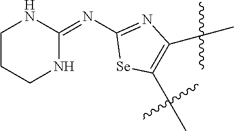 [SMILES: *c1nc(N=C2NCCCN2)[se]c1*] | 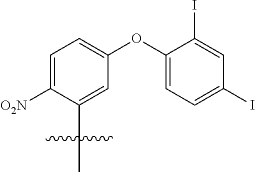 [SMILES: *c1cc(Oc2ccc(I)cc2I)ccc1[N+](=O)[O-]] | H |
| I-159 | 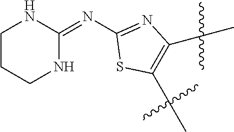 [SMILES: *c1nc(N=C2NCCCN2)sc1*] | 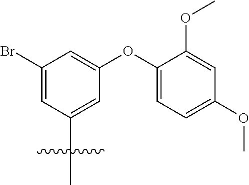 [SMILES: *c1cc(Br)cc(Oc2ccc(OC)cc2OC)c1] | H |
| I-160 | 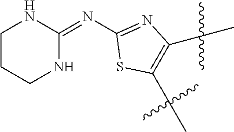 [SMILES: *c1nc(N=C2NCCCN2)sc1*] | 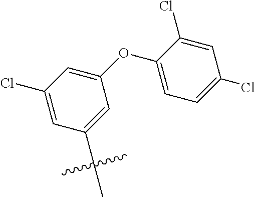 [SMILES: *c1cc(Cl)cc(Oc2ccc(Cl)cc2Cl)c1] | H |
| I-161 | 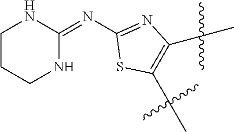 [SMILES: *c1nc(N=C2NCCCN2)sc1*] | 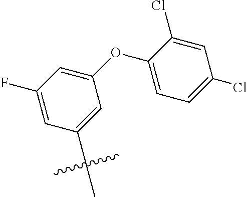 [SMILES: *c1cc(F)cc(Oc2ccc(Cl)cc2Cl)c1] | H |
| I-162 | 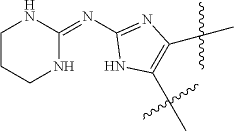 [SMILES: *c1nc(N=C2NCCCN2)[nH]c1*] | 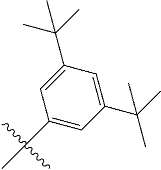 [SMILES: *c1cc(C(C)(C)C)cc(C(C)(C)C)c1] | Me |
| I-163 | 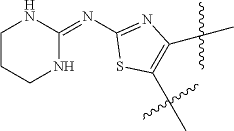 [SMILES: *c1nc(N=C2NCCCN2)sc1*] | 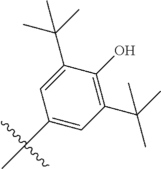 [SMILES: *c1cc(C(C)(C)C)c(O)c(C(C)(C)C)c1] | Cl |
| I-164 | 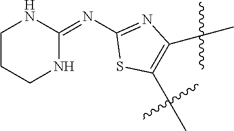 [SMILES: *c1nc(N=C2NCCCN2)sc1*] | 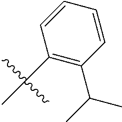 [SMILES: *c1ccccc1C(C)C] | Br |
| I-165 | 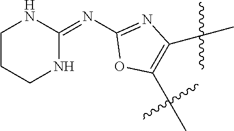 [SMILES: *c1nc(N=C2NCCCN2)oc1*] | 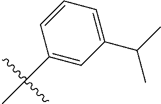 [SMILES: *c1cccc(C(C)C)c1] | Et |
| I-166 | 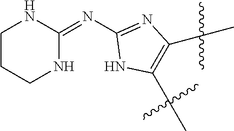 [SMILES: *c1nc(N=C2NCCCN2)[nH]c1*] | 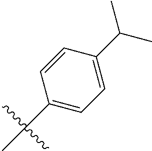 [SMILES: *c1ccc(C(C)C)cc1] | H |
| I-167 | 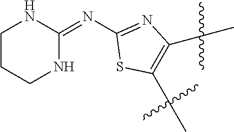 [SMILES: *c1nc(N=C2NCCCN2)sc1*] | 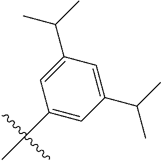 [SMILES: *c1cc(C(C)C)cc(C(C)C)c1] | Me |
| I-168 | 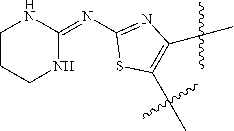 [SMILES: *c1nc(N=C2NCCCN2)sc1*] | 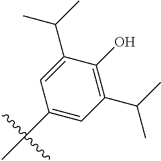 [SMILES: *c1cc(C(C)C)c(O)c(C(C)C)c1] | Cl |
| I-169 | 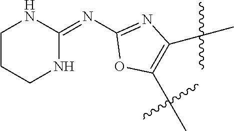 [SMILES: *c1nc(N=C2NCCCN2)oc1*] | 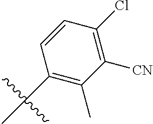 [SMILES: *c1ccc(Cl)c(C#N)c1C] | H |
| I-170 | 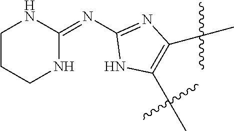 [SMILES: *c1nc(N=C2NCCCN2)[nH]c1*] | 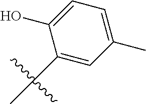 [SMILES: *c1cc(C)ccc1O] | H |
| I-171 | 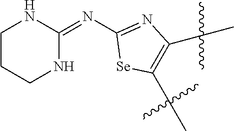 [SMILES: *c1nc(N=C2NCCCN2)[se]c1*] | 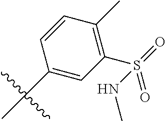 [SMILES: *c1ccc(C)c(S(=O)(=O)NC)c1] | H |
| I-172 | 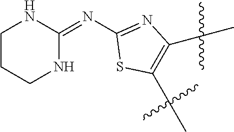 [SMILES: *c1nc(N=C2NCCCN2)sc1*] | 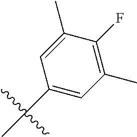 [SMILES: *c1cc(C)c(F)c(C)c1] | Et |
| I-173 | 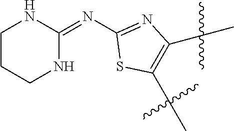 [SMILES: *c1nc(N=C2NCCCN2)sc1*] | 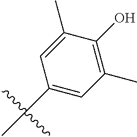 [SMILES: *c1cc(C)c(O)c(C)c1] | H |
| I-174 | 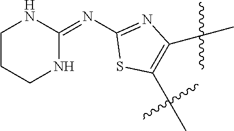 [SMILES: *c1nc(N=C2NCCCN2)sc1*] | 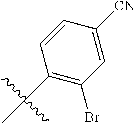 [SMILES: *c1ccc(C#N)cc1Br] | H |
| I-175 | 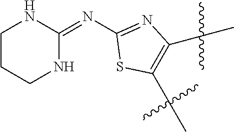 [SMILES: *c1nc(N=C2NCCCN2)sc1*] | 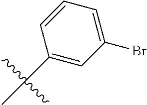 [SMILES: *c1cccc(Br)c1] | Cl |
| I-176 | 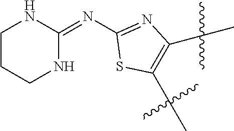 [SMILES: *c1nc(N=C2NCCCN2)sc1*] | 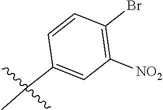 [SMILES: *c1ccc(Br)c([N+](=O)[O-])c1] | Br |
| I-177 | 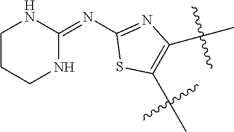 [SMILES: *c1nc(N=C2NCCCN2)sc1*] | 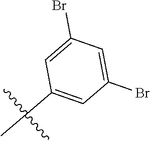 [SMILES: *c1cc(Br)cc(Br)c1] | F |
| I-178 | 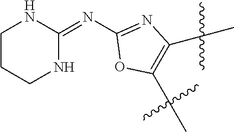 [SMILES: *c1nc(N=C2NCCCN2)oc1*] | 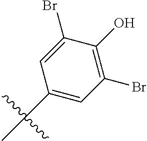 [SMILES: *c1cc(Br)c(O)c(Br)c1] | H |
| I-179 | 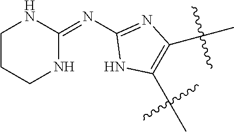 [SMILES: *c1nc(N=C2NCCCN2)[nH]c1*] | 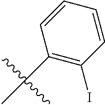 [SMILES: *c1ccccc1I] | H |
| I-180 | 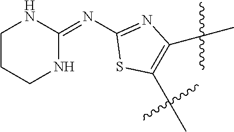 [SMILES: *c1nc(N=C2NCCCN2)sc1*] | 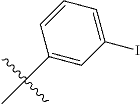 [SMILES: *c1cccc(I)c1] | H |
| I-181 | 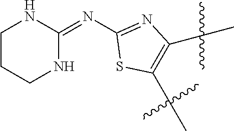 [SMILES: *c1nc(N=C2NCCCN2)sc1*] | 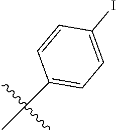 [SMILES: *c1ccc(I)cc1] | H |
| I-182 | 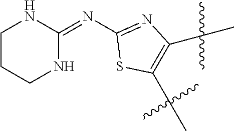 [SMILES: *c1nc(N=C2NCCCN2)sc1*] | 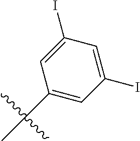 [SMILES: *c1cc(I)cc(I)c1] | Me |
| I-183 | 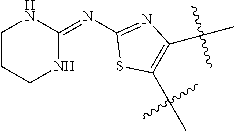 [SMILES: *c1nc(N=C2NCCCN2)sc1*] | 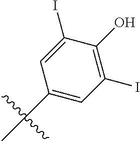 [SMILES: *c1cc(I)c(O)c(I)c1] | Cl |
| I-184 | 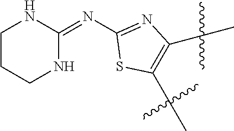 [SMILES: *c1nc(N=C2NCCCN2)sc1*] | 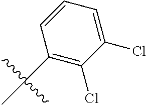 [SMILES: *c1cccc(Cl)c1Cl] | H |
| I-185 | 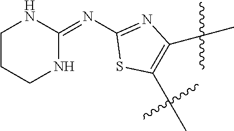 [SMILES: *c1nc(N=C2NCCCN2)sc1*] | 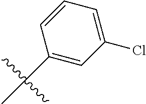 [SMILES: *c1cccc(Cl)c1] | Br |
| I-186 | 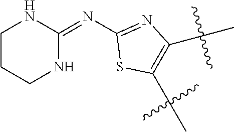 [SMILES: *c1nc(N=C2NCCCN2)sc1*] | 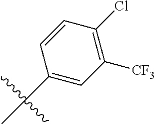 [SMILES: *c1ccc(Cl)c(C(F)(F)F)c1] | Cl |
| I-187 | 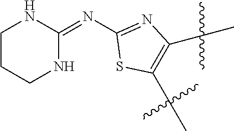 [SMILES: *c1nc(N=C2NCCCN2)sc1*] | 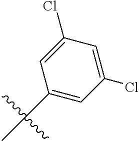 [SMILES: *c1cc(Cl)cc(Cl)c1] | H |
| I-188 | 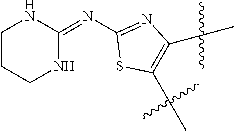 [SMILES: *c1nc(N=C2NCCCN2)sc1*] | 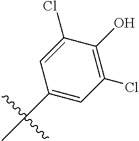 [SMILES: *c1cc(Cl)c(O)c(Cl)c1] | H |
| I-189 | 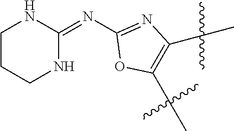 [SMILES: *c1nc(N=C2NCCCN2)oc1*] | 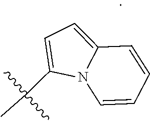 [SMILES: *c1ccc2ccccn12] | H |
| I-190 | 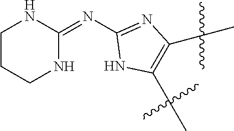 [SMILES: *c1nc(N=C2NCCCN2)[nH]c1*] | 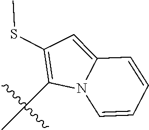 [SMILES: *c1c(SC)cc2ccccn12] | H |
| I-191 | 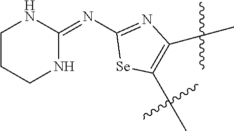 [SMILES: *c1nc(N=C2NCCCN2)[se]c1*] | 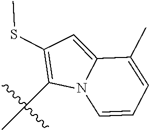 [SMILES: *c1c(SC)cc2c(C)cccn12] | H |
| I-192 | 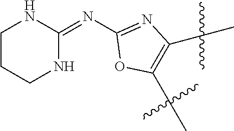 [SMILES: *c1nc(N=C2NCCCN2)oc1*] | 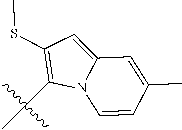 | H |
| I-193 | 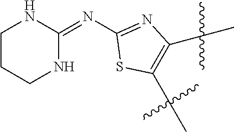 [SMILES: *c1nc(N=C2NCCCN2)sc1*] | 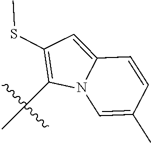 | Et |
| I-194 | 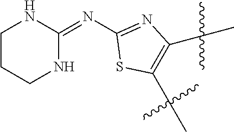 [SMILES: *c1nc(N=C2NCCCN2)sc1*] | 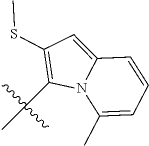 [SMILES: *c1c(SC)cc2cccc(C)n12] | H |
| I-195 | 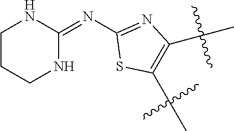 [SMILES: *c1nc(N=C2NCCCN2)sc1*] | 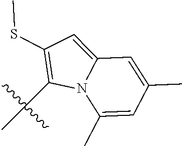 | Me |
| I-196 | 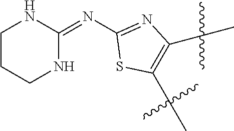 [SMILES: *c1nc(N=C2NCCCN2)sc1*] | 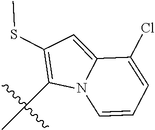 [SMILES: *c1c(SC)cc2c(Cl)cccn12] | Cl |
| I-197 | 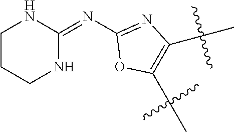 [SMILES: *c1nc(N=C2NCCCN2)oc1*] | 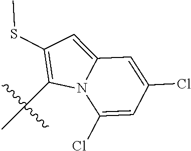 [SMILES: *c1c(SC)cc2cc(Cl)cc(Cl)n12] | Et |
| I-198 | 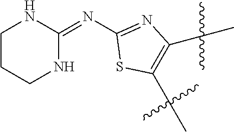 [SMILES: *c1nc(N=C2NCCCN2)sc1*] | 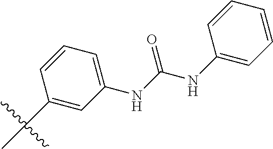 [SMILES: *c1cccc(NC(=O)Nc2ccccc2)c1] | Br |
| I-199 | 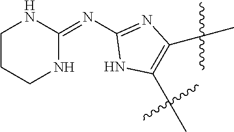 [SMILES: *c1nc(N=C2NCCCN2)[nH]c1*] | 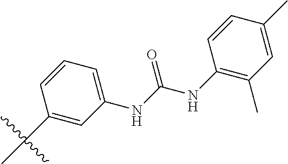 [SMILES: *c1cccc(NC(=O)Nc2ccc(C)cc2C)c1] | H |
| I-200 | 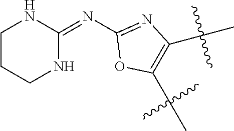 [SMILES: *c1nc(N=C2NCCCN2)oc1*] | 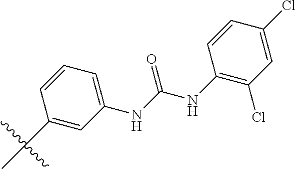 [SMILES: *c1cccc(NC(=O)Nc2ccc(Cl)cc2Cl)c1] | H |

As a pharmacologically active compound, the compound of the present invention can obviously be used as a medicament. Therefore, in addition to various properties tested in the examples, the compounds of the present invention can also possess various activities inherent to a medicament, for example, in vivo activity, bioavailability, druggability, toxicity, differential inhibitory activity, and so on. Based on the teachings of the present invention and routine technical means in the art, a skilled person will know how to obtain various compounds within the scope of the present invention and detect the various activities of such compounds; in other words, based on the teachings of the present invention and routine technical means in the art, a skilled person will know how to repeat, verify, and implement the present invention.

A skilled person will know that, to increase the solubility of a compound, the compound may be prepared as a pharmaceutically acceptable salt. Examples of the pharmaceutically acceptable salt of the compound of the present invention include, but are not limited to, inorganic and organic acid salts, such as hydrochlorides, hydrobromides, sulfates, citrates, lactates, tartrates, maleates, fumarates, mandelates, and oxalates; and inorganic and organic base salts formed with bases, such as sodium hydroxide, tris(hydroxymethyl)aminomethane (TRIS, trometamol), and N-methylglucosamine.

Based on the teachings of the present invention, a skilled person will know that the compound of the present invention may be administered in combination with one or more antibiotics to enhance the therapeutic efficacy of the antibiotics. The compound of the present invention may be administered to a subject in need thereof simultaneously with or sequentially with the antibiotics. In a preferred embodiment, the compound of the present invention may be administered simultaneously with the antibiotics to a subject in need thereof. Accordingly, based on the compound of the present invention, the present invention further provides a pharmaceutical composition comprising the compound of the present invention, an optical isomer, cis-trans isomer, or pharmaceutically acceptable salt thereof, or a combination thereof, and an antibiotic, as well as a pharmaceutically acceptable excipient. In a preferred embodiment, the pharmaceutical composition is in the form of a kit.

The pharmaceutical composition of the present invention comprises a safe and effective amount of the compound of the present invention, an optical isomer, cis-trans isomer, or pharmaceutically acceptable salt thereof, or a combination thereof. Herein, the term “safe and effective amount” refers to an amount of the compound that is sufficient to produce desired effects, such as a significant improvement in the condition, without causing serious side effects. Generally, the safe and effective amount of the active ingredient in the pharmaceutical composition can be determined by a clinician based on actual needs, such as the patient's age, sex, body weight, severity of the disease, previous treatment history, etc. Preferably, the aforementioned “one dose” refers to one capsule or tablet.

“Pharmaceutically acceptable carrier” refers to one or more compatible solid or liquid fillers or gel substances that are suitable for human use and must possess sufficient purity and sufficiently low toxicity. “Compatible” as used herein means that the components in the composition can be mixed with the compound of the present invention and with each other without significantly reducing the efficacy of the compound. Examples of pharmaceutically acceptable carriers include, but are not limited to, cellulose and its derivatives (such as sodium carboxymethyl cellulose, sodium ethyl cellulose, cellulose acetate, etc.), gelatin, talc, solid lubricants (such as stearic acid, magnesium stearate), calcium sulfate, vegetable oils (such as soybean oil, sesame oil, peanut oil, olive oil, etc.), polyols (such as propylene glycol, glycerin, mannitol, sorbitol, etc.), emulsifiers (such as Tween®), wetting agents (such as sodium lauryl sulfate), colorants, flavoring agents, stabilizers, antioxidants, preservatives, pyrogen-free water, and the like.

The administration method for the compounds or pharmaceutical compositions of the present invention is not particularly limited. Representative administration methods include (but are not limited to): oral, parenteral (intravenous, intramuscular, or subcutaneous) administration.

Solid dosage forms for oral administration include capsules, tablets, pills, powders, and granules. In such solid dosage forms, the active compound is mixed with at least one conventional inert excipient (or carrier), such as sodium citrate or dicalcium phosphate, or with the following components: (a) fillers or extenders, e.g., starch, lactose, sucrose, glucose, mannitol, and silicic acid; (b) binders, e.g., hydroxymethylcellulose, alginates, gelatin, polyvinylpyrrolidone, sucrose, and acacia; (c) humectants, e.g., glycerol; (d) disintegrating agents, e.g., agar, calcium carbonate, potato starch or tapioca starch, alginic acid, certain complex silicates, and sodium carbonate; (e) retarding agents, e.g., paraffin; (f) absorption accelerators, e.g., quaternary ammonium compounds; (g) wetting agents, e.g., cetyl alcohol and glyceryl monostearate; (h) adsorbents, e.g., kaolin; and (i) lubricants, e.g., talc, calcium stearate, magnesium stearate, solid polyethylene glycol, sodium lauryl sulfate, or mixtures thereof. In the case of capsules, tablets, and pills, the dosage form may also contain buffering agents.

Solid dosage forms, such as tablets, sugar-coated pills, capsules, pills, and granules can be prepared using coatings and shell materials, such as enteric coatings and other materials well known in the art. They may contain opacifying agents, and the release of the active compound or compounds in such compositions may be delayed so as to release at a certain part of the digestive tract. Examples of embedding components that can be used include polymeric substances and waxes. If necessary, the active compound may also be prepared into micro-capsules with one or more of the above-mentioned excipients.

Liquid dosage forms for oral administration include pharmaceutically acceptable emulsions, solutions, suspensions, syrups, or tinctures. In addition to the active compound, the liquid dosage forms may contain inert diluents commonly used in the art, such as water or other solvents, solubilizing agents and emulsifiers, e.g., ethanol, isopropanol, ethyl carbonate, ethyl acetate, propylene glycol, 1,3-butylene glycol, dimethylformamide, and oils, particularly cottonseed oil, groundnut oil, corn germ oil, olive oil, castor oil, and sesame oil or a mixture thereof.

In addition to these inert diluents, the composition may also include adjuvants, such as wetting agents, emulsifying and suspending agents, sweetening agents, flavoring agents, and perfuming agents.

Suspensions may contain suspending agents, for example, ethoxylated isostearyl alcohol, polyoxyethylene sorbitol and sorbitan esters, microcrystalline cellulose, aluminum metahydroxide, agar, and tragacanth or a mixture thereof, in addition to the active compound.

Compositions for parenteral injection may comprise physiologically acceptable sterile aqueous or non-aqueous solutions, dispersions, suspensions or emulsions, and sterile powders for reconstitution into sterile injectable solutions or dispersions. Suitable aqueous and non-aqueous carriers, diluents, solvents or excipients include water, ethanol, polyols (e.g., glycerol, propylene glycol, polyethylene glycol, etc.), and a suitable mixture thereof.

The compounds of the present invention may be administered alone or in combination with other pharmaceutically acceptable compounds.

### Advantages of the Present Invention

1. In the present invention, a new class of polymyxin synergists has been discovered, which exhibit high activities as antibiotic synergists, thereby demonstrating extremely superior antibacterial synergistic effects;
2. The polymyxin synergists of the present invention can be used to treat bacterial infections, particularly those associated with Escherichia coli, Acinetobacter baumannii, Klebsiella pneumoniae and Pseudomonas aeruginosa;
3. The use of the polymyxin synergists of the present invention can effectively reduce the dosage of polymyxins, thereby significantly lowering the risk of polymyxin toxicity during treatment; and
4. The polymyxin synergists of the present invention enable the treatment of infections caused by Escherichia coli, Acinetobacter baumannii, Klebsiella pneumoniae and Pseudomonas aeruginosa that are insensitive to antibiotics or exhibit weak antibacterial activity.

The technical solution of the present invention is further described below in conjunction with specific examples. However, following examples do not constitute limitations on the present invention. All application methods that adopt various approaches based on the principles and technical means of the present invention shall fall within the scope of the present invention. For experimental methods for which no specific conditions are specified in the following examples, conventional conditions are generally followed, or conditions recommended by the manufacturer are used. Unless otherwise stated, percentages and parts are calculated by weight.

### Example 1. Synthesis of 2-(4-(2-phenoxyphenyl)thiazol-2-yl)guanidine, i.e., Compound I-1

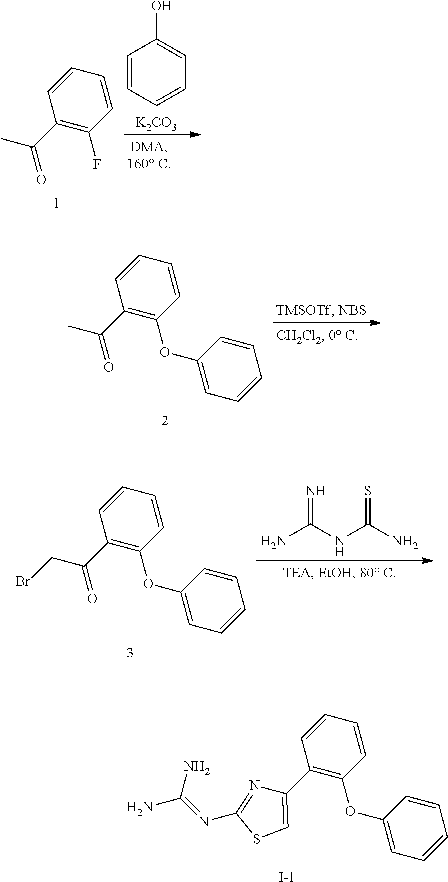

In a round-bottom flask, o-fluoroacetophenone 1 (1.38 g, 10 mmol), phenol (1.13 g, 12 mmol), and potassium carbonate (2.76 g, 20 mmol) were added, and dissolved in 30 mL of N,N-dimethylacetamide. The reaction was carried out at 160° C. for 24 hours. After cooling to room temperature, water and ethyl acetate were added to the mixture. The organic layer was washed with brine, dried over anhydrous magnesium sulfate, and concentrated. The magnesium sulfate was filtered off, and the filtrate was concentrated under a reduced pressure. The residue was purified by silica gel column chromatography (n-heptane-ethyl acetate=100:0 to 95:5) to obtain 2 (0.848 g, 40%) as a pale yellow oil.

¹H NMR (400 MHz, CDCl₃) δ 7.83 (1H, dd, J=7.8, 1.8 Hz), 7.34 (1H, ddd, J=8.7, 6.9, 1.5 Hz), 6.99-7.10 (3H, m), 6.81 (1H, d, J=8.2 Hz), 6.66 (1H, dd, J=8.3, 0.8 Hz), 2.71 (3H, s).

To a solution of 2 (441 mg, 2.08 mmol) and triethylamine (0.346 mL, 2.50 mmol) in dichloromethane (5 mL) was added TMSOTf (0.451 mL, 2.50 mmol) at 0° C. The mixture was allowed to reach room temperature. At 0° C., NBS (370 mg, 2.08 mmol) was added to the reaction mixture and stirred for 1 hour. The organic layer was washed with brine, dried over anhydrous sodium sulfate, and concentrated under a reduced pressure. The residue was purified by silica gel column chromatography (n-heptane-ethyl acetate=90:10 to 85:15) to afford a pale yellow oil 3 (484 mg, 80%).

¹H NMR (400 MHz, CDCl₃) δ 7.91 (1H, dd, J=7.9, 1.8 Hz), 7.36-7.39 (1H, m), 7.03-7.10 (3H, m), 6.87 (1H, d, J=8.1 Hz), 6.62 (1H, dd, J=8.4, 0.8 Hz), 4.72 (2H, s).

3 (291 mg, 1 mmol) was dissolved in 15 mL of anhydrous ethanol, add guanidinethiourea (118 mg, 1 mmol), and refluxed at 80° C. for 1 hour. After the reaction is completed, the mixture was colled, and concentrated under a reduced pressure. The residue was purified by silica gel column chromatography (dichloromethane-methanol=95:5 to 90:10) to obtain a white solid I-1 (186 mg, 60%).

White solid; yield 60%; melting point: 235.2-238.1° C.; ¹H NMR (400 MHz, DMSO-d₆) δ 8.04 (dd, J=7.7, 1.8 Hz, 1H), 7.36 (td, J=7.5, 1.9 Hz, 2H), 7.33-7.22 (m, 2H), 7.10 (d, J=4.4 Hz, 2H), 7.00-6.94 (m, 3H), 6.84 (s, 3H). ¹³C NMR (101 MHz, DMSO-d6) δ 174.34, 157.48, 157.39, 153.28, 145.28, 130.46, 128.99, 127.26, 124.83, 123.33, 121.04, 117.91, 107.99. HRMS (EI+): m/z C₁₆H₁₄N₄OS calculated: 310.0888, measured: 310.0891.

### Example 2. Synthesis of Compound I-2

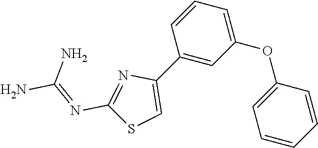 [SMILES: NC(N)=Nc1nc(-c2cccc(Oc3ccccc3)c2)cs1]

Using suitable starting materials, compound I-2 was synthesized following similar procedures as described for compound I-1.

¹H NMR (400 MHz, DMSO-d6) δ 7.62 (d, J=7.9 Hz, 1H), 7.51-7.47 (m, 1H), 7.44-7.36 (m, 3H), 7.22 (s, 1H), 7.17-7.12 (m, 1H), 7.07-7.01 (m, 2H), 7.00-6.75 (m, 4H). 13C NMR (101 MHz, DMSO-d6) δ 175.58, 157.54, 157.32, 157.22, 148.93, 137.32, 130.62, 130.50, 121.18, 118.97, 117.79, 116.35, 104.59. HRMS (EI+): m/z C₁₆H₁₄N₄OS calculated: 310.0888, measured: 310.0891.

### Example 3. Synthesis of Compound I-8

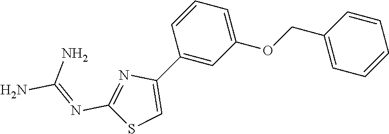 [SMILES: NC(N)=Nc1nc(-c2cccc(OCc3ccccc3)c2)cs1]

Using suitable starting materials, compound I-8 was synthesized following similar procedures as described for compound I-1.

¹H NMR (400 MHz, DMSO-d₆) δ 7.69 (s, 4H), 7.57 (s, 1H), 7.53-7.48 (m, 4H), 7.43 (d, J=7.2 Hz, 2H), 7.35 (dt, J=6.9, 3.0 Hz, 2H), 7.00 (dd, J=8.1, 1.8 Hz, 1H), 5.19 (s, 2H). ¹³C NMR (101 MHz, DMSO) δ 166.54, 159.12, 155.66, 149.83, 137.58, 135.62, 130.28, 128.94 (2C), 128.32, 128.23 (2C), 118.89, 114.73, 112.65, 107.11, 69.74. HRMS (EI+): m/z C₁₇H₁₆N₄OS calculated: 324.1045; measured: 324.1041.

### Example 4. Synthesis of Compound I-14

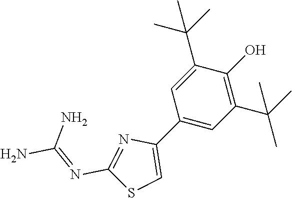 [SMILES: CC(C)(C)c1cc(-c2csc(N=C(N)N)n2)cc(C(C)(C)C)c1O]

Using suitable starting materials, compound I-14 was synthesized following similar procedures as described for compound I-1.

¹H NMR (400 MHz, DMSO-d₆) δ 7.82 (s, 4H), 7.55 (s, 2H), 7.30 (s, 1H), 7.14 (s, 1H), 1.43 (s, 18H). ¹³C NMR (101 MHz, DMSO) δ 165.27, 155.58, 154.61, 151.15, 139.79 (2C), 126.03, 122.90 (2C), 104.49, 35.10 (2C), 30.81 (6C). HRMS (EI+): m/z C₁₈H₂₆N₄S calculated: 346.1827, measured: 346.1830.

### Example 5. Synthesis of Compound I-44

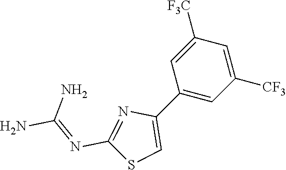 [SMILES: NC(N)=Nc1nc(-c2cc(C(F)(F)F)cc(C(F)(F)F)c2)cs1]

Using suitable starting materials, compound I-44 was synthesized following similar procedures as described for compound I-1.

¹H NMR (400 MHz, DMSO-d₆) δ 9.46 (s, 2H), 8.60 (s, 1H), 8.22 (d, J=24 Hz, 1H), 8.14 (d, J=24 Hz, 1H), 8.08 (s, 1H). ¹³C NMR (101 MHz, DMSO) δ 162.55, 154.58, 147.34, 136.25, 131.34 ((q, ²J_{CF}=32.32 Hz, 2C), 126.96 (q, ³J_{CF}=3.03 Hz, 2C), 123.81 (q, ¹J_{CF}=271.69 Hz, 2C), 121.67 (q, ³J_{CF}=3.03 Hz), 112.56. HRMS (EI+): m/z C₁₃H₁₆N₄S calculated: 354.0374, measured: 354.0372.

### Example 6. Synthesis of Compound I-47

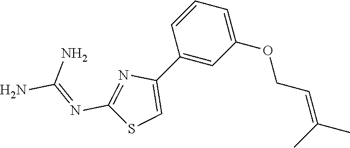 [SMILES: CC(C)=CCOc1cccc(-c2csc(N=C(N)N)n2)c1]

Using suitable starting materials, compound I-47 was synthesized following similar procedures as described for compound I-1.

¹H NMR (400 MHz, DMSO-d₆) δ 7.41 (d, J=7.7 Hz, 1H), 7.36 (s, 1H), 7.29 (t, J=7.9 Hz, 1H), 7.21 (s, 1H), 6.86 (dd, J=8.1, 2.0 Hz, 1H), 5.52-5.41 (m, 1H), 4.63-4.54 (m, 2H), 1.79-1.73 (m, 6H). ¹³C NMR (101 MHz, DMSO-d₆) δ 174.85, 159.19, 157.35, 149.66, 137.48, 136.62, 130.06, 120.60, 118.40, 113.88, 112.27, 104.27, 64.74, 25.96, 18.57. HRMS (EI+): m/z C₁₃H₁₆N₄S calculated: 302.1201, measured: 302.1197.

### Example 7. Synthesis of Compound I-51

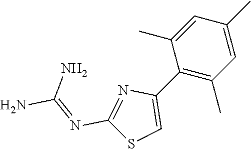 [SMILES: Cc1cc(C)c(-c2csc(N=C(N)N)n2)c(C)c1]

Using suitable starting materials, compound I-51 was synthesized following similar procedures as described for compound I-1.

¹H NMR (400 MHz, DMSO-d₆) δ 7.96 (s, 1H), 7.81 (s, 3H), 6.84 (s, 2H), 2.24 (s, 3H), 2.13 (s, 6H). ¹³C NMR (101 MHz, DMSO-d₆) δ 172.69, 165.40, 140.70, 137.21, 133.02, 128.29, 95.90, 19.15 (3C). HRMS (EI+): m/z C₁₃H₁₆N₄S calculated: 260.1096, measured: 260.1054.

### Example 8. Synthesis of Compound I-56

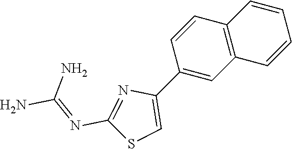 [SMILES: NC(N)=Nc1nc(-c2ccc3ccccc3c2)cs1]

Using suitable starting materials, compound I-56 was synthesized following similar procedures as described for compound I-1.

¹H NMR (400 MHz, DMSO-d₆) δ 8.40 (s, 1H), 8.00 (dd, J=13.1, 8.5 Hz, 2H), 7.95-7.80 (m, 2H), 7.51 (t, J=7.2 Hz, 2H), 7.34 (s, 1H), 7.03 (s, 3H). ¹³C NMR (101 MHz, DMSO-d₆) δ 175.75, 157.55, 149.75, 133.78, 132.82, 132.75, 128.59, 128.46, 128.04, 126.80, 126.28, 124.51, 124.39, 104.57. HRMS (EI+): m/z C₁₄H₁₂N₄S calculated: 268.0783, measured: 268.0780.

### Example 9. Synthesis of Compound I-74

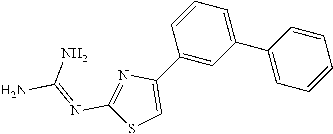 [SMILES: NC(N)=Nc1nc(-c2cccc(-c3ccccc3)c2)cs1]

Using suitable starting materials, compound I-74 was synthesized following similar procedures as described for compound I-1.

¹H NMR (400 MHz, DMSO-d₆) δ 8.14 (s, 1H), 7.93 (d, J=7.7 Hz, 1H), 7.83-7.71 (m, 6H), 7.64 (d, J=7.7 Hz, 1H), 7.53 (q, J=7.9 Hz, 3H), 7.42 (t, J=7.3 Hz, 1H). ¹³C NMR (101 MHz, DMSO-d₆) δ 166.43, 155.61, 150.00, 141.16, 140.49, 134.91, 129.84, 129.41, 128.08, 127.39, 126.82, 125.55, 124.39, 107.32. HRMS (EI+): m/z C₁₆H₁₄N₄S calculated: 284.0939, measured: 268.0737.

### Example 10. Synthesis of Compound I-82

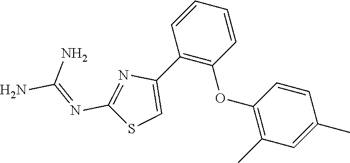 [SMILES: Cc1ccc(Oc2ccccc2-c2csc(N=C(N)N)n2)c(C)c1]

Using suitable starting materials, compound I-82 was synthesized following similar procedures as described for compound I-1.

¹H NMR (400 MHz, CDCl₃) δ 7.96 (dd, J=7.6, 1.9 Hz, 1H), 7.32 (s, 1H), 7.10-7.00 (m, 3H), 6.91-6.85 (m, 1H), 6.72 (d, J=8.2 Hz, 1H), 6.63 (dd, J=8.0, 1.2 Hz, 1H), 2.25 (s, 3H), 2.14 (s, 3H). ¹³C NMR (101 MHz, DMSO) δ 174.21, 157.48, 154.92, 152.04, 145.46, 133.49, 132.49, 130.15, 129.03, 128.70, 128.31, 125.34, 123.19, 119.45, 117.38, 107.89, 20.76, 16.21. HRMS (EI+): m/z C₁₈H₁₈N₄OS calculated: 338.1201, measured: 338.1208.

### Example 11. Synthesis of Compound I-85

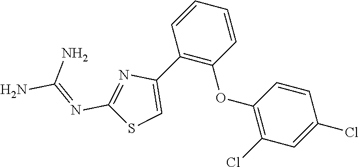 [SMILES: NC(N)=Nc1nc(-c2ccccc2Oc2ccc(Cl)cc2Cl)cs1]

Using suitable starting materials, compound I-85 was synthesized following similar procedures as described for compound I-1.

¹H NMR (400 MHz, DMSO-d₆) δ 8.13 (dd, J=7.7, 1.6 Hz, 1H), 8.03-7.73 (m, 5H), 7.53 (s, 1H), 7.43 (dd, J=8.8, 2.5 Hz, 1H), 7.38 (td, J=7.9, 1.7 Hz, 1H), 7.33-7.27 (m, 1H), 7.02 (d, J=8.8 Hz, 1H), 6.91 (d, J=8.1 Hz, 1H). ¹³C NMR (101 MHz, DMSO) δ 155.24, 153.14, 151.08, 145.31, 130.84, 130.68, 130.10, 129.39, 128.77, 125.57, 125.09, 125.02, 121.53, 118.92, 111.58. HRMS (EI+): m/z C₁₆H₁₂Cl₂N₄OS calculated: 378.0109, measured: 378.0106.

### Example 12. Synthesis of Compound I-86

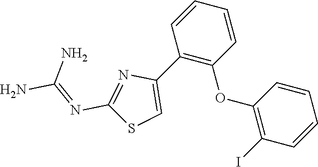 [SMILES: NC(N)=Nc1nc(-c2ccccc2Oc2ccccc2I)cs1]

Using suitable starting materials, compound I-86 was synthesized following similar procedures as described for compound I-1.

¹H NMR (400 MHz, DMSO-d₆) δ 8.16 (d, J=7.6 Hz, 1H), 8.06 (s, 4H), 7.97 (d, J=7.8 Hz, 1H), 7.68 (s, 1H), 7.42 (t, J=7.6 Hz, 1H), 7.37 (s, 1H), 7.30-7.22 (m, 1H), 7.02 (t, J=7.6 Hz, 1H), 6.95 (d, J=8.0 Hz, 1H), 6.79-6.72 (m, 1H). ¹³C NMR (101 MHz, DMSO) δ 161.68, 155.58, 154.83, 153.90, 145.58, 140.31, 130.79, 130.06 (2C), 126.74, 124.35, 120.02 (2C), 118.22, 112.26, 90.26. HRMS (EI+): m/z C₁₆H₁₃IN₄OS calculated: 435.9855, measured: 435.9858.

### Example 13. Synthesis of Compound I-104

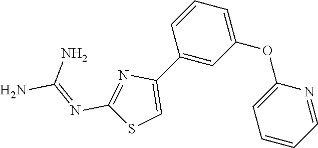 [SMILES: NC(N)=Nc1nc(-c2cccc(Oc3ccccn3)c2)cs1]

Using suitable starting materials, compound I-104 was synthesized following similar procedures as described for compound I-1.

¹H NMR (400 MHz, DMSO-d₆) δ 9.29 (s, 2H), 8.21-8.06 (m, 5H), 7.87 (td, J=8.4, 2.0 Hz, 1H), 7.81 (d, J=9.7 Hz, 2H), 7.72 (s, 1H), 7.47 (t, J=7.9 Hz, 1H), 7.12 (m, 2H), 7.05 (d, J=8.3 Hz, 1H). ¹³C NMR (101 MHz, DMSO-d₆) δ 163.53, 154.73, 154.31, 149.61, 147.92, 140.74, 137.40, 135.29, 130.58, 122.76, 121.80, 119.56, 119.33, 111.94, 109.51. HRMS (EI+): m/z C₁₅H₁₃N₅OS calculated: 311.0841, measured: 311.0844.

### Example 14. Synthesis of Compound I-106

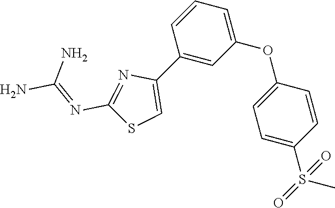 [SMILES: CS(=O)(=O)c1ccc(Oc2cccc(-c3csc(N=C(N)N)n3)c2)cc1]

Using suitable starting materials, compound I-106 was synthesized following similar procedures as described for compound I-1.

¹H NMR (400 MHz, DMSO-d₆) δ 8.22 (s, 4H), 7.92 (s, 4H), 7.83 (s, 1H), 7.56 (t, J=7.8 Hz, 1H), 7.20 (d, J=8.1 Hz, 2H), 7.15 (d, J=7.8 Hz, 1H), 3.22 (s, 3H). ¹³C NMR (101 MHz, DMSO) δ 162.00, 160.80, 155.46, 154.37, 149.33, 136.00, 135.19, 131.40, 130.19, 123.41, 120.64, 118.56, 117.91, 110.02, 44.38. HRMS (EI+): m/z C₁₇H₁₆N₄O₃S₂ calculated: 388.0664, measured: 388.0666.

### Example 15. Synthesis of Compound I-107

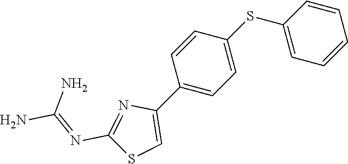 [SMILES: NC(N)=Nc1nc(-c2ccc(Sc3ccccc3)cc2)cs1]

Using suitable starting materials, compound I-107 was synthesized following similar procedures as described for compound I-1.

¹H NMR (400 MHz, DMSO-d₆) δ 7.86 (d, J=8.0 Hz, 2H), 7.39 (d, J=8.0 Hz, 2H), 7.35 (m, 5H), 7.22 (s, 1H), 6.93 (s, 3H). ¹³C NMR (101 MHz, DMSO-d₆) δ 175.73, 157.53, 148.94, 135.67 (2C), 134.61 (2C), 133.49 (2C), 130.06, 127.77, 127.08 (2C), 104.42. HRMS (EI+): m/z C₁₆H₁₄N₄S₂ calculated: 326.0660, measured: 326.0663.

### Example 16. Synthesis of Compound I-109

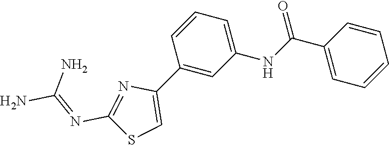 [SMILES: NC(N)=Nc1nc(-c2cccc(NC(=O)c3ccccc3)c2)cs1]

Using suitable starting materials, compound I-109 was synthesized following similar procedures as described for compound I-1.

¹H NMR (400 MHz, DMSO-d₆) δ 10.30 (s, 1H), 8.20 (s, 1H), 7.99 (d, J=7.5 Hz, 2H), 7.78 (d, J=8.0 Hz, 1H), 7.59 (dd, J=17.3, 6.6 Hz, 4H), 7.38 (t, J=7.9 Hz, 1H), 7.11 (s, 1H), 6.98 (s, 3H). ¹³C NMR (101 MHz, DMS-d₆O) δ 175.47, 166.12, 157.51, 149.68, 139.93, 135.76, 135.51, 132.08, 129.29, 128.91 (2C), 128.19 (2C), 121.41, 119.85, 118.22, 103.82. HRMS (EI+): m/z C₁₇H₁₅N₅OS calculated: 337.0997, measured: 337.1001.

### Example 17. Synthesis of Compound I-111

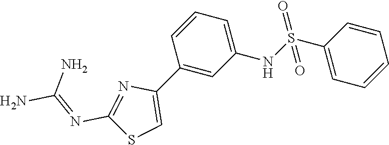 [SMILES: NC(N)=Nc1nc(-c2cccc(NS(=O)(=O)c3ccccc3)c2)cs1]

Using suitable starting materials, compound I-111 was synthesized following similar procedures as described for compound I-1.

¹H NMR (400 MHz, DMSO-d₆) δ 7.82 (d, J=7.0 Hz, 2H), 7.65-7.56 (m, 4H), 7.55-7.38 (m, 5H), 7.29-7.22 (m, 2H), 7.08-7.01 (m, 1H). ¹³C NMR (101 MHz, DMSO-d₆) δ 182.03, 170.16, 167.20, 156.50, 149.20, 139.98, 138.62, 135.51, 133.49, 129.97, 129.82, 127.21, 121.71, 119.41, 117.71, 106.12. HRMS (EI+): m/z C₁₆H₁₅N₅O₂S₂ calculated: 373.0667, measured: 373.0672.

### Example 18. Synthesis of Compound I-112

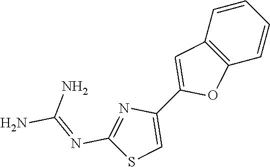 [SMILES: NC(N)=Nc1nc(-c2cc3ccccc3o2)cs1]

Using suitable starting materials, compound I-112 was synthesized following similar procedures as described for compound I-1.

¹H NMR (400 MHz, DMSO-d₆) δ 8.26 (s, 4H), 7.74 (s, 1H), 7.70 (d, J=7.5 Hz, 1H), 7.64 (d, J=8.1 Hz, 1H), 7.57 (s, 1H), 7.38 (t, J=8.1 Hz, 1H), 7.31 (t, J=7.5 Hz, 1H). ¹³C NMR (101 MHz, DMSO) δ 161.64, 154.78, 154.38, 151.00, 141.41, 128.77, 125.66, 124.01, 122.06, 111.68, 110.59, 104.73. HRMS (EI+): m/z C₁₂H₁₀N₄OS calculated: 258.0575, measured: 258.0577.

### Example 19. Synthesis of Compound I-113

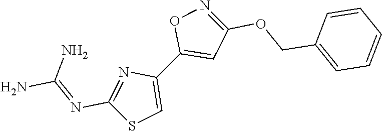 [SMILES: NC(N)=Nc1nc(-c2cc(OCc3ccccc3)no2)cs1]

Using suitable starting materials, compound I-113 was synthesized following similar procedures as described for compound I-1.

¹H NMR (400 MHz, DMSO-d₆) δ 7.50 (m, 2H), 7.41 (m, 4H), 7.13 (s, 3H), 6.68 (s, 1H), 5.30 (s, 2H). ¹³C NMR (101 MHz, DMSO) δ 172.14, 166.34, 157.27, 138.46, 136.40, 128.95 (2C), 128.83, 128.70 (2C), 109.86, 92.14, 71.72. HRMS (EI+): m/z C₁₄H₁₃N₅O₂S calculated: 315.0790, measured: 315.0793.

### Example 20. Synthesis of Compound I-114

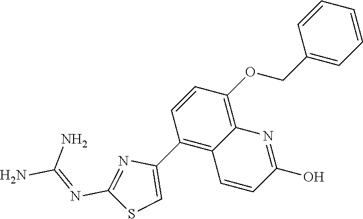 [SMILES: NC(N)=Nc1nc(-c2ccc(OCc3ccccc3)c3nc(O)ccc23)cs1]

Using suitable starting materials, compound I-114 was synthesized following similar procedures as described for compound I-1.

¹H NMR (400 MHz, DMSO-d₆) δ 10.76 (s, 1H), 8.27 (d, J=9.9 Hz, 1H), 7.60 (d, J=7.3 Hz, 2H), 7.40 (t, J=7.4 Hz, 2H), 7.33 (t, J=7.2 Hz, 1H), 7.24 (s, 2H), 6.88 (s, 4H), 6.82 (s, 1H), 6.56 (d, J=9.9 Hz, 1H), 5.36 (s, 2H). ¹³C NMR (101 MHz, DMSO-d₆) δ 175.43, 161.49, 157.45, 148.50, 144.21, 138.80, 137.11, 130.07, 128.86 (2C), 128.40, 128.26 (2C), 127.16, 122.81, 122.67, 117.70, 112.82, 106.71, 70.35. HRMS (EI+): m/z C₁₄H₁₃N₅O₂S calculated: 391.1103, measured: 391.1100.

### Example 21. Synthesis of Compound I-115

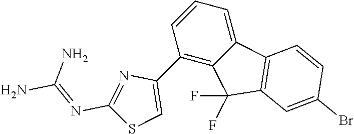 [SMILES: NC(N)=Nc1nc(-c2cccc3c2C(F)(F)c2cc(Br)ccc2-3)cs1]

Using suitable starting materials, compound I-115 was synthesized following similar procedures as described for compound I-1.

¹H NMR (400 MHz, DMSO-d₆) δ 8.16 (s, 1H), 8.09 (d, J=8.0 Hz, 1H), 7.96 (s, 1H), 7.86 (d, J=8.0 Hz, 1H), 7.82 (s, 2H), 7.44 (s, 1H), 6.95 (s, 4H). ¹³C NMR (101 MHz, DMSO-d₆) δ 163.53, 154.74, 143.55, 131.89, 131.06, 129.27, 128.11, 127.80, 127.51, 126.89, 126.42, 125.60, 124.30, 124.10, 117.09, 113.51. HRMS (EI+): m/z C₁₇H₁₁BrF₂N₄S calculated: 419.9856, measured: 419.9860.

### Example 22. Synthesis of Compound I-117

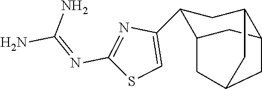 [SMILES: NC(N)=Nc1nc(C2CC3CC4CC3CC2C4)cs1]

Using suitable starting materials, compound I-117 was synthesized following similar procedures as described for compound I-1.

¹H NMR (400 MHz, DMSO-d₆) δ 7.85 (s, 4H), 6.66 (s, 1H), 2.02 (m, 3H), 1.86 (m, 6H), 1.72 (m, 6H). ¹³C NMR (101 MHz, DMSO-d₆) δ 164.10, 161.57, 155.47, 103.92, 46.17, 41.82 (3C), 36.70 (3C), 36.43, 28.39 (2C). HRMS (EI+): m/z C₁₄H₂₀N₄S calculated: 276.1409, measured: 276.1406.

### Example 23. Synthesis of Compound I-118

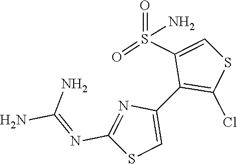 [SMILES: NC(N)=Nc1nc(-c2c(S(N)(=O)=O)csc2Cl)cs1]

Using suitable starting materials, compound I-118 was synthesized following similar procedures as described for compound I-1.

¹H NMR (400 MHz, DMSO-d₆) δ 7.74 (s, 1H), 7.63 (s, 2H), 7.47 (s, 1H), 6.80 (s, 4H). ¹³C NMR (101 MHz, DMSO) δ 175.27, 157.17, 142.92, 136.59, 135.34, 131.26, 129.99, 111.05. HRMS (EI+): m/z C₈H₈ClN₅O₂S calculated: 336.9529, measured: 336.9523.

### Example 24. Synthesis of Compound I-121

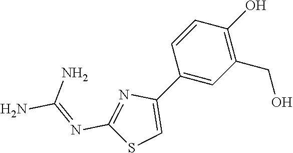 [SMILES: NC(N)=Nc1nc(-c2ccc(O)c(CO)c2)cs1]

Using suitable starting materials, compound I-121 was synthesized following similar procedures as described for compound I-1.

¹H NMR (400 MHz, DMSO-d₆) δ 7.78 (s, 1H), 7.56 (d, J=7.9 Hz, 1H), 7.20 (s, 4H), 6.96 (s, 1H), 6.82 (d, J=8.2 Hz, 1H), 4.95 (s, 1H), 4.54 (s, 2H). ¹³C NMR (101 MHz, DMSO-d₆) δ 172.65, 156.92, 154.44, 150.49, 129.03, 126.29, 125.49, 125.47, 115.18, 101.42, 58.80. HRMS (EI+): m/z C₁₁H₁₂N₄O₂S calculated: 264.0681, measured: 264.0679.

### Example 25. Synthesis of Compound I-123

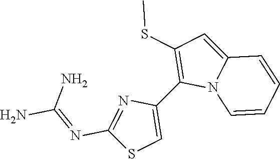 [SMILES: CSc1cc2ccccn2c1-c1csc(N=C(N)N)n1]

Using suitable starting materials, compound I-123 was synthesized following similar procedures as described for compound I-1.

¹H NMR (400 MHz, DMSO-d₆) δ 8.51 (d, J=7.1 Hz, 1H), 7.42 (d, J=8.8 Hz, 1H), 6.92 (d, J=42.4 Hz, 5H), 6.83-6.74 (m, 1H), 6.62 (t, J=6.8 Hz, 1H), 6.53 (s, 1H), 2.45 (s, 3H). ¹³C NMR (101 MHz, DMSO-d₆) δ 162.12, 154.68, 147.58, 144.28, 140.38, 134.98, 128.54, 123.04, 119.35, 118.74, 116.39, 15.59. HRMS (EI+): m/z C₁₃H₁₃N₅S₂ calculated: 203.0612, measured: 203.0615.

### Example 26. Synthesis of Compound I-131

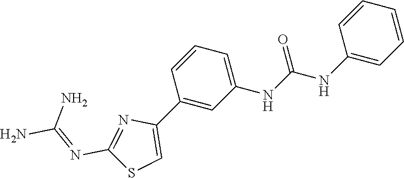 [SMILES: NC(N)=Nc1nc(-c2cccc(NC(=O)Nc3ccccc3)c2)cs1]

Using suitable starting materials, compound I-131 was synthesized following similar procedures as described for compound I-1.

¹H NMR (400 MHz, DMSO-d₆) δ 8.77 (s, 1H), 8.65 (s, 1H), 7.76-7.72 (m, 2H), 7.46-7.42 (m, 3H), 7.39-7.34 (m, 2H), 7.26-7.20 (m, 2H), 6.97 (s, 1H), 6.92 (tt, J=7.5, 2.0 Hz, 1H), 6.82 (s, 2H). ¹³C NMR (101 MHz, DMSO-d₆) δ 160.08, 155.52, 154.49, 146.26, 139.19, 138.69, 131.07, 129.81, 129.05, 126.55, 123.70, 122.67, 119.94, 112.54, 110.97. HRMS (EI+): m/z C₁₇H₁₆N₆OS calculated: 352.1106, measured: 352.1108.

### Example 27. Synthesis of Compound I-143

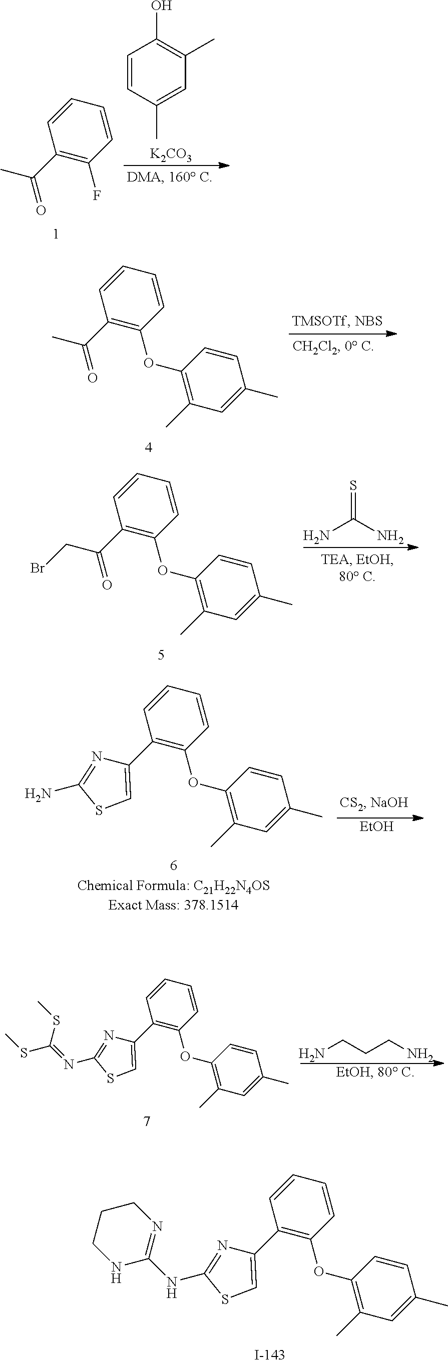

Using suitable starting materials and following similar procedures as described for compound I-1, the intermediate compound 6 was synthesized. To a solution of intermediate 6 (434 mg, 1.37 mmol) and CS₂ in DMF (2 mL), 10 N aqueous NaOH (0.29 mL, 2.9 mmol) was added, and the mixture was stirred at room temperature for 30 minutes. At 0° C., Mel (616 μL, 9.86 mmol) was added to the mixture, which was then stirred at the same temperature for 4 hours. The extract was washed with brine, dried over Na₂SO₄, and concentrated under a reduced pressure. The residue was purified by silica gel column chromatography (hexane-EtOAc=50:1 to 10:1) to afford 7 (274 mg, 68%) as a colorless solid.

To a solution of 7 (30 mg, 0.075 mmol) in 1,4-dioxane (2 mL) was added propanediamine (45 mg, 0.749 mmol). The mixture was stirred at 100° C. for 2 hours, then cooled to room temperature and concentrated under a reduced pressure. The residue was purified by silica gel column chromatography (hexane-EtOAc=9:1 to 1:1) to afford I-142 (16 mg, 59%) as a white solid.

¹H NMR (400 MHz, DMSO-d₆) δ 8.26 (s, 2H), 8.01 (dd, J=7.6, 1.8 Hz, 1H), 7.19 (dd, J=7.9, 1.8 Hz, 1H), 7.16-7.11 (m, 2H), 7.10 (s, 1H), 7.00 (dd, J=8.3, 1.9 Hz, 1H), 6.73 (d, J=8.2 Hz, 1H), 6.64 (dd, J=8.0, 1.1 Hz, 1H), 3.30 (d, J=8.8 Hz, 4H), 2.27 (s, 3H), 2.15 (s, 3H), 1.82 (p, J=5.8 Hz, 2H). ¹³C NMR (101 MHz, DMSO-d₆) δ 158.78, 155.84, 154.49, 153.91, 145.71, 133.03, 132.23, 130.50, 129.48, 128.60, 127.20, 125.24, 124.40, 116.33, 110.90, 47.24, 41.09, 27.93, 20.49, 15.95. HRMS (EI+): m/z C₂₁H₂₂N₄OS calculated: 378.1514, measured: 378.1517.

### Example 28. Synthesis of Compound I-163

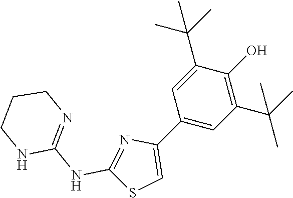 [SMILES: CC(C)(C)c1cc(-c2csc(NC3=NCCCN3)n2)cc(C(C)(C)C)c1O]

Using suitable starting materials, compound I-163 was synthesized following similar procedures as described for compound I-143.

¹H NMR (400 MHz, DMSO-d₆) δ 7.82 (s, 4H), 7.55 (s, 2H), 7.30 (s, 1H), 7.14 (s, 1H), 3.30 (d, J=8.8 Hz, 4H), 1.82 (p, J=5.8 Hz, 2H), 1.43 (s, 18H). ¹³C NMR (101 MHz, DMSO) δ 165.27, 155.58, 154.61, 151.15, 139.79 (2C), 126.03, 122.90 (2C), 104.49, 41.09, 35.10 (2C), 30.81 (6C), 27.93, 20.49. HRMS (EI+): m/z C₁₈H₂₆N₄S calculated: 386.2140, measured: 386.2142.

### Example 29. Test on Activities

Antibacterial Synergistic Activity Test on the Compounds of the Present Invention

### Experimental Method

Polymyxin E was diluted in sterile MH broth medium to concentrations of 16, 8, 4, 2, 1, 0.5, 0.25, 0.125, 0.0625, 0.031, and 0.015 μg/ml, and the drug to be tested was diluted to concentrations of 256, 128, 64, 32, 16, 8, and 4 μg/ml. According to the checkerboard method, combinations of the drug to be tested and polymyxin at different concentrations in ascending order were placed into a 96-well plate, and corresponding blank control groups were set up. 50 μl of the drug solution was added to each well, then 100 μl of bacterial liquid was added, and incubated in a 37° C. incubator for 16 hours. Antibacterial effects were observed with naked eyes and the MIC values were recorded. Calculation and judgment criteria:

Fractional Inhibitory Concentration (FIC) is calculated as follows:

$$FIC index = ( MIC of Drug A in combination / MIC of Drug A alone ) + ( MIC of Drug B in combination / MIC of Drug B alone ) .$$

Judgment criteria: FIC≤0.5: synergistic effect; 0.5<FIC<1: additive effect; 1<FIC<2: indifferent effect; FIC≥2: antagonistic effect.

### Experimental Results

1. Results of the test on the antibacterial synergistic activities of the compound of Formula I according to the present invention on polymyxin E indicate that the compounds of the present invention exhibit significant antibacterial synergistic activity on polymyxin B. The FIC values are shown in the table below.

In the table: ATCC19606 strain is Acinetobacter baumannii, ATCC25922 strain is Escherichia coli, ATCC13883 strain is Klebsiella pneumoniae, ATCC9027 strain is Pseudomonas aeruginosa. All four strains were purchased from ATCC.

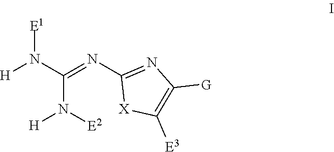

TABLE 1
Synergistic activity (FIC value) of the compound of Formula I

| Number | ATCC19606 | ATCC25922 | ATCC13883 | ATCC9027 |
|---|---|---|---|---|
| I-1 | 1.25 | 1.25 | 0.53 | 0.52 |
| I-2 | 0.26 | 0.51 | 0.75 | 0.53 |
| I-3 | 1.25 | 0.5 | 1.25 | 1.25 |
| I-4 | 1.25 | 0.5 | 1.25 | 1.25 |
| I-5 | 1.25 | 1.25 | 1.25 | 1.25 |
| I-6 | 0.75 | 0.5 | 1.25 | 1.25 |
| I-7 | 0.16 | 0.51 | 0.51 | 0.75 |
| I-8 | 0.25 | 1.25 | 0.53 | 0.53 |
| I-9 | 0.13 | 0.26 | 0.16 | 0.31 |
| I-10 | 1.25 | 0.5 | 1.25 | 1.25 |
| I-11 | 0.3 | 0.5 | 0.25 | 0.63 |
| I-12 | 0.28 | 0.5 | 0.25 | 0.63 |
| I-13 | 0.15 | 0.39 | 0.33 | 0.62 |
| I-14 | 0.13 | 0.38 | 0.31 | 0.53 |
| I-15 | 1.25 | 0.5 | 1.25 | 1.25 |
| I-16 | 0.3 | 0.5 | 0.25 | 0.63 |
| I-17 | 0.28 | 0.5 | 0.25 | 0.63 |
| I-18 | 0.15 | 0.39 | 0.33 | 0.62 |
| I-19 | 0.12 | 0.38 | 0.31 | 0.31 |
| I-20 | 0.75 | 0.5 | 0.63 | 0.56 |
| I-21 | 0.52 | 0.63 | 0.52 | 1.25 |
| I-22 | 0.75 | 0.5 | 0.38 | 0.63 |
| I-23 | 0.52 | 0.63 | 0.52 | 1.25 |
| I-24 | 0.5 | 1.25 | 1.25 | 1.25 |
| I-25 | 0.13 | 0.38 | 0.31 | 0.53 |
| I-26 | 0.5 | 0.63 | 1.25 | 1.25 |
| I-27 | 0.51 | 1.25 | 1.25 | 1.25 |
| I-28 | 1.25 | 1.25 | 1.25 | 0.51 |
| I-29 | 0.26 | 1.25 | 1.25 | 1.25 |
| I-30 | 0.13 | 0.52 | 0.19 | 1.25 |
| I-31 | 0.25 | 0.56 | 0.31 | 0.5 |
| I-32 | 0.25 | 1.25 | 1.25 | 0.52 |
| I-33 | 0.28 | 0.5 | 0.25 | 0.63 |
| I-34 | 0.75 | 1.25 | 1.25 | 1.25 |
| I-35 | 0.13 | 0.52 | 0.19 | 1.25 |
| I-36 | 0.25 | 0.56 | 0.31 | 0.5 |
| I-37 | 0.25 | 1.25 | 1.25 | 0.52 |
| I-38 | 0.28 | 0.5 | 0.25 | 0.63 |
| I-39 | 0.75 | 1.25 | 1.25 | 1.25 |
| I-40 | 0.13 | 0.52 | 0.19 | 0.51 |
| I-41 | 0.5 | 0.38 | 0.38 | 0.63 |
| I-42 | 1.25 | 0.53 | 1.25 | 1.25 |
| I-43 | 1.25 | 0.5 | 1.25 | 0.63 |
| I-44 | 0.14 | 0.31 | 0.25 | 0.52 |
| I-45 | 0.13 | 0.52 | 0.19 | 1.25 |
| I-46 | 0.31 | 0.27 | 0.25 | 0.52 |
| I-47 | 0.75 | 0.5 | 1.25 | 1.25 |
| I-48 | 0.16 | 0.51 | 0.51 | 0.75 |
| I-49 | 0.25 | 1.25 | 0.53 | 0.53 |
| I-50 | 0.13 | 0.26 | 0.16 | 0.31 |
| I-51 | 1.25 | 0.5 | 1.25 | 1.25 |
| I-52 | 0.3 | 0.5 | 0.25 | 0.63 |
| I-53 | 0.28 | 0.5 | 0.25 | 0.63 |
| I-54 | 0.15 | 0.39 | 0.33 | 0.62 |
| I-55 | 0.13 | 0.38 | 0.31 | 0.53 |
| I-56 | 1.25 | 0.5 | 1.25 | 1.25 |
| I-57 | 0.3 | 0.5 | 0.25 | 0.63 |
| I-58 | 0.28 | 0.5 | 0.25 | 0.63 |
| I-59 | 0.15 | 0.39 | 0.33 | 0.62 |
| I-60 | 0.12 | 0.38 | 0.31 | 0.31 |
| I-61 | 0.75 | 0.5 | 0.63 | 0.56 |
| I-62 | 0.52 | 0.63 | 0.52 | 1.25 |
| I-63 | 0.75 | 0.5 | 0.38 | 0.63 |
| I-64 | 0.52 | 0.63 | 0.52 | 1.25 |
| I-65 | 0.5 | 1.25 | 1.25 | 1.25 |
| I-66 | 0.13 | 0.38 | 0.31 | 0.53 |
| I-67 | 0.5 | 0.63 | 1.25 | 1.25 |
| I-68 | 0.51 | 1.25 | 1.25 | 1.25 |
| I-69 | 1.25 | 1.25 | 1.25 | 0.51 |
| I-70 | 0.26 | 1.25 | 1.25 | 1.25 |
| I-71 | 0.13 | 0.52 | 0.19 | 1.25 |
| I-72 | 0.25 | 0.56 | 0.31 | 0.5 |
| I-73 | 0.25 | 1.25 | 1.25 | 0.52 |
| I-74 | 0.15 | 0.39 | 0.33 | 0.62 |
| I-75 | 0.12 | 0.38 | 0.31 | 0.31 |
| I-76 | 0.75 | 0.5 | 0.63 | 0.56 |
| I-77 | 0.52 | 0.63 | 0.52 | 1.25 |
| I-78 | 0.75 | 0.5 | 0.38 | 0.63 |
| I-79 | 0.52 | 0.63 | 0.52 | 1.25 |
| I-80 | 0.52 | 0.63 | 0.52 | 1.25 |
| I-81 | 0.31 | 0.14 | 0.09 | 0.06 |
| I-82 | 0.03 | 0.18 | 0.16 | 0.28 |
| I-83 | 0.63 | 0.27 | 0.09 | 0.04 |
| I-84 | 0.63 | 0.27 | 0.09 | 0.04 |
| I-85 | 0.04 | 0.26 | 0.19 | 0.52 |
| I-86 | 0.38 | 0.04 | 0.19 | 0.04 |
| I-87 | 0.03 | 0.18 | 0.16 | 0.28 |
| I-88 | 0.04 | 0.2 | 0.13 | 0.28 |
| I-89 | 0.02 | 0.15 | 0.13 | 0.18 |
| I-90 | 0.03 | 0.15 | 0.13 | 0.18 |
| I-91 | 0.04 | 0.26 | 0.19 | 0.52 |
| I-92 | 0.38 | 0.04 | 0.19 | 0.04 |
| I-93 | 0.03 | 0.18 | 0.16 | 0.28 |
| I-94 | 0.04 | 0.26 | 0.19 | 0.52 |
| I-95 | 0.38 | 0.04 | 0.19 | 0.04 |
| I-96 | 0.03 | 0.18 | 0.16 | 0.28 |
| I-97 | 0.04 | 0.2 | 0.13 | 0.28 |
| I-98 | 0.31 | 0.14 | 0.09 | 0.06 |
| I-99 | 0.03 | 0.18 | 0.16 | 0.28 |
| I-100 | 0.02 | 0.15 | 0.13 | 0.18 |
| I-101 | 0.25 | 1.25 | 1.25 | 0.52 |
| I-102 | 1.25 | 1.25 | 1.25 | 0.51 |
| I-103 | 1.25 | 0.53 | 1.25 | 1.25 |
| I-104 | 0.25 | 1.25 | 1.25 | 0.52 |
| I-105 | 1.25 | 1.25 | 1.25 | 0.51 |
| I-106 | 1.25 | 0.53 | 1.25 | 1.25 |
| I-107 | 1.25 | 0.53 | 1.25 | 1.25 |
| I-108 | 0.03 | 0.18 | 0.16 | 0.28 |
| I-109 | 1.25 | 0.27 | 1.25 | 1.25 |
| I-110 | 0.25 | 0.56 | 0.31 | 0.5 |
| I-111 | 0.5 | 0.63 | 0.25 | 1.25 |
| I-112 | 0.51 | 1.25 | 1.25 | 0.52 |
| I-113 | 1.25 | 0.53 | 1.25 | 1.25 |
| I-114 | 0.27 | 0.5 | 1.25 | 1.25 |
| I-115 | 1.25 | 1.25 | 1.25 | 1.25 |
| I-116 | 0.25 | 0.5 | 0.63 | 1.25 |
| I-117 | 0.5 | 0.63 | 0.25 | 1.25 |
| I-118 | 1.25 | 1.25 | 0.19 | 1.25 |
| I-119 | 1.25 | 1.25 | 0.19 | 1.25 |
| I-120 | 1.25 | 0.75 | 0.28 | 0.25 |
| I-121 | 1.25 | 0.56 | 1.25 | 1.25 |
| I-122 | 0.56 | 0.38 | 0.56 | 0.75 |
| I-123 | 0.31 | 0.14 | 0.09 | 0.06 |
| I-124 | 0.38 | 0.04 | 0.19 | 0.04 |
| I-125 | 0.03 | 0.18 | 0.16 | 0.28 |
| I-126 | 0.04 | 0.2 | 0.13 | 0.28 |
| I-127 | 0.31 | 0.14 | 0.09 | 0.06 |
| I-128 | 0.03 | 0.18 | 0.16 | 0.28 |
| I-129 | 0.31 | 0.14 | 0.09 | 0.06 |
| I-130 | 0.03 | 0.18 | 0.16 | 0.28 |
| I-131 | 0.31 | 0.14 | 0.09 | 0.06 |
| I-132 | 0.03 | 0.18 | 0.16 | 0.28 |
| I-133 | 0.38 | 0.04 | 0.19 | 0.04 |
| I-134 | 1.25 | 1.25 | 0.53 | 0.52 |
| I-135 | 0.26 | 0.51 | 0.75 | 0.53 |
| I-136 | 1.25 | 0.5 | 1.25 | 1.25 |
| I-137 | 1.25 | 0.5 | 1.25 | 1.25 |
| I-138 | 1.25 | 1.25 | 1.25 | 1.25 |
| I-139 | 0.75 | 0.5 | 1.25 | 1.25 |
| I-140 | 0.51 | 1.25 | 1.25 | 0.52 |
| I-141 | 0.51 | 1.25 | 1.25 | 0.52 |
| I-142 | 0.31 | 0.14 | 0.09 | 0.06 |
| I-143 | 0.38 | 0.04 | 0.19 | 0.04 |
| I-144 | 0.03 | 0.18 | 0.16 | 0.28 |
| I-145 | 0.04 | 0.2 | 0.13 | 0.28 |
| I-146 | 0.31 | 0.14 | 0.09 | 0.06 |
| I-147 | 0.03 | 0.18 | 0.16 | 0.28 |
| I-148 | 0.31 | 0.14 | 0.09 | 0.06 |
| I-149 | 0.03 | 0.18 | 0.16 | 0.28 |
| I-150 | 0.31 | 0.14 | 0.09 | 0.06 |
| I-151 | 0.03 | 0.18 | 0.16 | 0.28 |
| I-152 | 0.38 | 0.04 | 0.19 | 0.04 |
| I-153 | 0.31 | 0.14 | 0.09 | 0.06 |
| I-154 | 0.38 | 0.04 | 0.19 | 0.04 |
| I-155 | 0.03 | 0.18 | 0.16 | 0.28 |
| I-156 | 0.04 | 0.2 | 0.13 | 0.28 |
| I-157 | 0.31 | 0.14 | 0.09 | 0.06 |
| I-158 | 0.03 | 0.18 | 0.16 | 0.28 |
| I-159 | 0.31 | 0.14 | 0.09 | 0.06 |
| I-160 | 0.03 | 0.18 | 0.16 | 0.28 |
| I-161 | 0.31 | 0.14 | 0.09 | 0.06 |
| I-162 | 0.03 | 0.18 | 0.16 | 0.28 |
| I-163 | 0.38 | 0.04 | 0.19 | 0.04 |
| I-164 | 0.15 | 0.39 | 0.33 | 0.62 |
| I-165 | 0.13 | 0.38 | 0.31 | 0.53 |
| I-166 | 1.25 | 0.5 | 1.25 | 1.25 |
| I-167 | 0.3 | 0.5 | 0.25 | 0.63 |
| I-168 | 0.28 | 0.5 | 0.25 | 0.63 |
| I-169 | 0.15 | 0.39 | 0.33 | 0.62 |
| I-170 | 0.12 | 0.38 | 0.31 | 0.31 |
| I-171 | 0.75 | 0.5 | 0.63 | 0.56 |
| I-172 | 0.52 | 0.63 | 0.52 | 1.25 |
| I-173 | 0.75 | 0.5 | 0.38 | 0.63 |
| I-174 | 0.52 | 0.63 | 0.52 | 1.25 |
| I-175 | 0.5 | 1.25 | 1.25 | 1.25 |
| I-176 | 0.13 | 0.38 | 0.31 | 0.53 |
| I-177 | 0.5 | 0.63 | 1.25 | 1.25 |
| I-178 | 0.51 | 1.25 | 1.25 | 1.25 |
| I-179 | 1.25 | 1.25 | 1.25 | 0.51 |
| I-180 | 0.26 | 1.25 | 1.25 | 1.25 |
| I-181 | 0.13 | 0.52 | 0.19 | 1.25 |
| I-182 | 0.25 | 0.56 | 0.31 | 0.5 |
| I-183 | 0.25 | 1.25 | 1.25 | 0.52 |
| I-184 | 0.28 | 0.5 | 0.25 | 0.63 |
| I-185 | 0.75 | 1.25 | 1.25 | 1.25 |
| I-186 | 0.13 | 0.52 | 0.19 | 1.25 |
| I-187 | 0.25 | 0.56 | 0.31 | 0.5 |
| I-188 | 0.25 | 1.25 | 1.25 | 0.52 |
| I-189 | 0.28 | 0.5 | 0.25 | 0.63 |
| I-190 | 0.75 | 1.25 | 1.25 | 1.25 |
| I-191 | 0.13 | 0.52 | 0.19 | 0.51 |
| I-192 | 0.5 | 0.38 | 0.38 | 0.63 |
| I-193 | 1.25 | 0.53 | 1.25 | 1.25 |
| I-194 | 1.25 | 0.5 | 1.25 | 0.63 |
| I-195 | 0.14 | 0.31 | 0.25 | 0.52 |
| I-196 | 0.13 | 0.52 | 0.19 | 1.25 |
| I-197 | 0.31 | 0.27 | 0.25 | 0.52 |
| I-198 | 0.75 | 0.5 | 1.25 | 1.25 |
| I-199 | 0.16 | 0.51 | 0.51 | 0.75 |
| I-200 | 0.25 | 1.25 | 0.53 | 0.53 |

### 2. Antibacterial Activities of the Compound Used Alone

The broth microdilution method was used to test the antibacterial effects of the compounds of the present invention against various Gram-negative bacterial strains when used alone. The results showed that most of the compounds did not exhibit inhibiting effects on the tested strains. The results are shown in the table below.

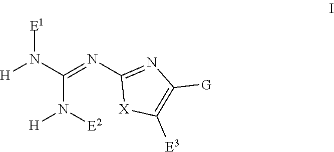

TABLE 2
Antibacterial activity of the compound of Formula I (μg/ml)

| Number | ATCC19606 | ATCC25922 | ATCC13883 | ATCC9027 |
|---|---|---|---|---|
| I-1 | 256 | 256 | 256 | 256 |
| I-2 | 256 | 256 | 256 | 256 |
| I-3 | 256 | 256 | 256 | 256 |
| I-4 | 256 | 256 | 256 | 256 |
| I-5 | 256 | 256 | 256 | 256 |
| I-6 | 256 | 256 | 256 | 256 |
| I-7 | 256 | 256 | 256 | 256 |
| I-8 | 256 | 256 | 256 | 128 |
| I-9 | 256 | 256 | 256 | 256 |
| I-10 | 256 | 256 | 256 | 256 |
| I-11 | 256 | 256 | 256 | 256 |
| I-12 | 256 | 256 | 256 | 256 |
| I-13 | 256 | 256 | 256 | 256 |
| I-14 | 256 | 256 | 256 | 256 |
| I-15 | 256 | 256 | 256 | 256 |
| I-16 | 256 | 256 | 256 | 256 |
| I-17 | 256 | 256 | 256 | 256 |
| I-18 | 256 | 256 | 256 | 256 |
| I-19 | 256 | 256 | 256 | 256 |
| I-20 | 256 | 256 | 256 | 256 |
| I-21 | 256 | 128 | 128 | 64 |
| I-22 | 256 | 256 | 256 | 256 |
| I-23 | 256 | 256 | 256 | 256 |
| I-24 | 256 | 256 | 256 | 256 |
| I-25 | 256 | 256 | 256 | 256 |
| I-26 | 256 | 256 | 256 | 256 |
| I-27 | 256 | 256 | 256 | 256 |
| I-28 | 256 | 256 | 256 | 256 |
| I-29 | 256 | 256 | 256 | 256 |
| I-30 | 256 | 256 | 256 | 256 |
| I-31 | 256 | 256 | 256 | 256 |
| I-32 | 256 | 256 | 256 | 256 |
| I-33 | 256 | 256 | 256 | 256 |
| I-34 | 256 | 256 | 256 | 256 |
| I-35 | 256 | 256 | 256 | 256 |
| I-36 | 256 | 128 | 128 | 64 |
| I-37 | 256 | 256 | 256 | 256 |
| I-38 | 256 | 256 | 256 | 256 |
| I-39 | 256 | 128 | 128 | 64 |
| I-40 | 256 | 256 | 256 | 256 |
| I-41 | 256 | 256 | 256 | 256 |
| I-42 | 256 | 256 | 256 | 256 |
| I-43 | 256 | 256 | 256 | 256 |
| I-44 | 256 | 256 | 256 | 256 |
| I-45 | 256 | 256 | 256 | 256 |
| I-46 | 256 | 256 | 256 | 256 |
| I-47 | 256 | 256 | 256 | 256 |
| I-48 | 256 | 256 | 256 | 256 |
| I-49 | 256 | 256 | 256 | 256 |
| I-50 | 256 | 256 | 256 | 256 |
| I-51 | 256 | 256 | 256 | 256 |
| I-52 | 256 | 256 | 256 | 256 |
| I-53 | 256 | 256 | 256 | 256 |
| I-54 | 256 | 256 | 256 | 256 |
| I-55 | 256 | 256 | 256 | 256 |
| I-56 | 256 | 256 | 256 | 256 |
| I-57 | 256 | 256 | 256 | 256 |
| I-58 | 256 | 256 | 256 | 256 |
| I-59 | 256 | 256 | 256 | 256 |
| I-60 | 256 | 256 | 256 | 256 |
| I-61 | 256 | 256 | 256 | 256 |
| I-62 | 256 | 128 | 128 | 64 |
| I-63 | 256 | 256 | 256 | 256 |
| I-64 | 256 | 256 | 256 | 256 |
| I-65 | 256 | 256 | 256 | 256 |
| I-66 | 256 | 256 | 256 | 256 |
| I-67 | 256 | 256 | 256 | 256 |
| I-68 | 256 | 256 | 256 | 256 |
| I-69 | 256 | 256 | 256 | 256 |
| I-70 | 256 | 256 | 256 | 256 |
| I-71 | 256 | 256 | 256 | 256 |
| I-72 | 256 | 256 | 256 | 256 |
| I-73 | 256 | 256 | 256 | 256 |
| I-74 | 256 | 256 | 256 | 256 |
| I-75 | 256 | 256 | 256 | 256 |
| I-76 | 256 | 256 | 256 | 256 |
| I-77 | 256 | 256 | 256 | 256 |
| I-78 | 256 | 256 | 256 | 128 |
| I-79 | 256 | 256 | 256 | 256 |
| I-80 | 256 | 256 | 256 | 256 |
| I-81 | 256 | 256 | 256 | 256 |
| I-82 | 256 | 256 | 256 | 256 |
| I-83 | 256 | 256 | 256 | 256 |
| I-84 | 256 | 256 | 256 | 256 |
| I-85 | 256 | 256 | 256 | 256 |
| I-86 | 256 | 256 | 256 | 256 |
| I-87 | 256 | 256 | 256 | 256 |
| I-88 | 256 | 256 | 256 | 256 |
| I-89 | 256 | 256 | 256 | 256 |
| I-90 | 256 | 256 | 256 | 256 |
| I-91 | 256 | 128 | 128 | 64 |
| I-92 | 256 | 256 | 256 | 256 |
| I-93 | 256 | 256 | 256 | 256 |
| I-94 | 256 | 256 | 256 | 256 |
| I-95 | 256 | 256 | 256 | 256 |
| I-96 | 256 | 256 | 256 | 256 |
| I-97 | 256 | 256 | 256 | 256 |
| I-98 | 256 | 256 | 256 | 256 |
| I-99 | 256 | 256 | 256 | 256 |
| I-100 | 256 | 128 | 256 | 256 |
| I-101 | 256 | 256 | 256 | 256 |
| I-102 | 256 | 256 | 256 | 256 |
| I-103 | 256 | 256 | 256 | 256 |
| I-104 | 256 | 256 | 256 | 256 |
| I-105 | 256 | 256 | 256 | 256 |
| I-106 | 256 | 256 | 256 | 256 |
| I-107 | 256 | 256 | 256 | 256 |
| I-108 | 256 | 256 | 256 | 256 |
| I-109 | 256 | 256 | 256 | 256 |
| I-110 | 256 | 256 | 256 | 256 |
| I-111 | 256 | 256 | 256 | 256 |
| I-112 | 256 | 256 | 256 | 256 |
| I-113 | 256 | 128 | 128 | 64 |
| I-114 | 256 | 256 | 256 | 256 |
| I-115 | 256 | 256 | 256 | 256 |
| I-116 | 256 | 256 | 256 | 256 |
| I-117 | 256 | 32 | 64 | 16 |
| I-118 | 256 | 256 | 256 | 256 |
| I-119 | 256 | 256 | 256 | 256 |
| I-120 | 256 | 256 | 256 | 256 |
| I-121 | 256 | 256 | 256 | 256 |
| I-122 | 256 | 256 | 256 | 256 |
| I-123 | 256 | 256 | 256 | 256 |
| I-124 | 256 | 256 | 256 | 256 |
| I-125 | 256 | 256 | 256 | 256 |
| I-126 | 256 | 256 | 256 | 256 |
| I-127 | 256 | 128 | 128 | 64 |
| I-128 | 256 | 256 | 256 | 256 |
| I-129 | 256 | 256 | 256 | 256 |
| I-130 | 256 | 256 | 256 | 256 |
| I-131 | 256 | 256 | 256 | 256 |
| I-132 | 256 | 256 | 256 | 256 |
| I-133 | 256 | 256 | 256 | 256 |
| I-134 | 256 | 256 | 256 | 256 |
| I-135 | 256 | 256 | 256 | 256 |
| I-136 | 256 | 256 | 256 | 256 |
| I-137 | 256 | 128 | 128 | 128 |
| I-138 | 256 | 256 | 256 | 256 |
| I-139 | 256 | 256 | 256 | 256 |
| I-140 | 256 | 256 | 256 | 256 |
| I-141 | 256 | 256 | 256 | 256 |
| I-142 | 256 | 256 | 256 | 256 |
| I-143 | 256 | 256 | 256 | 256 |
| I-144 | 256 | 256 | 256 | 256 |
| I-145 | 256 | 256 | 256 | 256 |
| I-146 | 256 | 256 | 256 | 256 |
| I-147 | 256 | 128 | 128 | 64 |
| I-148 | 256 | 256 | 256 | 256 |
| I-149 | 256 | 256 | 256 | 256 |
| I-150 | 256 | 256 | 256 | 256 |
| I-151 | 256 | 256 | 256 | 256 |
| I-152 | 256 | 256 | 256 | 256 |
| I-153 | 256 | 256 | 256 | 256 |
| I-154 | 256 | 256 | 256 | 256 |
| I-155 | 256 | 128 | 128 | 64 |
| I-156 | 256 | 256 | 256 | 256 |
| I-157 | 256 | 256 | 256 | 256 |
| I-158 | 256 | 256 | 256 | 256 |
| I-159 | 256 | 256 | 256 | 256 |
| I-160 | 256 | 256 | 256 | 256 |
| I-161 | 256 | 256 | 256 | 256 |
| I-162 | 256 | 256 | 256 | 256 |
| I-163 | 256 | 128 | 128 | 64 |
| I-164 | 256 | 256 | 256 | 256 |
| I-165 | 256 | 256 | 256 | 256 |
| I-166 | 256 | 256 | 256 | 256 |
| I-167 | 256 | 256 | 256 | 256 |
| I-168 | 256 | 256 | 256 | 256 |
| I-169 | 256 | 256 | 256 | 256 |
| I-170 | 256 | 256 | 256 | 256 |
| I-171 | 256 | 256 | 256 | 256 |
| I-172 | 256 | 128 | 128 | 64 |
| I-173 | 256 | 256 | 256 | 256 |
| I-174 | 256 | 256 | 256 | 256 |
| I-175 | 256 | 256 | 256 | 256 |
| I-176 | 256 | 256 | 256 | 256 |
| I-177 | 256 | 256 | 256 | 256 |
| I-178 | 256 | 256 | 256 | 256 |
| I-179 | 256 | 128 | 128 | 64 |
| I-180 | 256 | 256 | 256 | 256 |
| I-181 | 256 | 256 | 256 | 256 |
| I-182 | 256 | 256 | 256 | 256 |
| I-183 | 256 | 256 | 256 | 256 |
| I-184 | 256 | 256 | 256 | 256 |
| I-185 | 256 | 256 | 256 | 256 |
| I-186 | 256 | 256 | 256 | 256 |
| I-187 | 256 | 128 | 128 | 64 |
| I-188 | 256 | 256 | 256 | 256 |
| I-189 | 256 | 256 | 256 | 256 |
| I-190 | 256 | 256 | 256 | 256 |
| I-191 | 256 | 256 | 256 | 256 |
| I-192 | 256 | 256 | 256 | 256 |
| I-193 | 256 | 256 | 256 | 256 |
| I-194 | 256 | 128 | 128 | 64 |
| I-195 | 256 | 256 | 256 | 256 |
| I-196 | 256 | 256 | 256 | 256 |
| I-197 | 256 | 256 | 256 | 256 |
| I-198 | 256 | 128 | 128 | 64 |
| I-199 | 256 | 256 | 256 | 256 |
| I-200 | 256 | 256 | 256 | 256 |

3. Synergistic Effect on Inhibiting Clinical Strains of Acinetobacter baumannii.

Compound I-82 of the present invention was used as the representative compound and tested against clinical strains of Acinetobacter baumannii. The results showed that 1-82 exhibited broad-spectrum synergistic effects against clinical strains of A. baumannii with various resistance profiles. The selected A. baumannii strains were isolated from hospital clinical samples and preserved in the laboratory for research purposes. Among them, the aba39 and aba pmrA strains are polymyxin E-resistant, with MIC values of polymyxin E as 64 μg/ml and 256 μg/ml, respectively. The synergistic effects (FIC) are shown in Table 3 below.

TABLE 3

| No. of strain | FIC |
|---|---|
| aba 30 | 0.08 |
| aba 39 | 0.13 |
| aba 1611 | 0.09 |
| aba 1613 | 0.13 |
| aba 1617 | 0.08 |
| aba 1638 | 0.02 |
| aba 1645 | 0.16 |
| aba 4201 | 0.09 |
| aba 4230 | 0.06 |
| aba 6359 | 0.09 |
| aba 6380 | 0.14 |
| aba 6382 | 0.09 |
| aba 6410 | 0.06 |
| aba mia | 0.05 |
| aba pmrA | 0.16 |

### 4. In Vivo Activity Test:

Representative compounds I-14 and I-82 were tested for their in vivo synergistic bactericidal activities when combined with polymyxin E. The specific method is as follows:

Normal Balb/c mice (6-8 weeks old) were used, and the animals were randomly divided into four groups (5 mice per group):
I: Control group
II: Polymyxin E at 0.5 mg/kg body weight
III: Polymyxin E at 0.5 mg/kg body weight+Compound I-14 at 16 mg/kg
IV: Polymyxin E at 0.5 mg/kg body weight+Compound I-82 at 16 mg/kg

Freshly cultured ATCC19606 strains (logarithmic growth phase) were washed with saline, adjusted to an OD600 of ~1.0, and mixed at 1:1 (v/v) with 10% porcine mucin. Each mouse was intraperitoneally administered 500 μl of the mixture. 1 hour later, an intraperitoneal injection was given again, with the control group receiving only saline. Mortality was observed at 96 hours.

The experimental results are as follows:

By Day 5 of the experiment, all mice in Group I and Group II died, whereas the survival rate in Group III remained at 80%, and that in Group IV remained above 60%. There was no significant difference between Group I and Group II, indicating that a dosage of 0.5 mg/kg Polymyxin E had no therapeutic effects on the bacteremia in mice caused by Acinetobacter baumannii. In contrast, Compounds I-14 and I-82 exhibited synergistic effects with Polymyxin E in vivo.

All documents cited in the present invention are incorporated herein by reference as if each individual document were specifically and individually incorporated by reference. Furthermore, it should be understood that after reading the above teachings of the present invention, a skilled person may make various changes or modifications to the present invention, and such equivalent forms shall fall within the scope defined by the claims appended to this application, either.

1. A compound of Formula I, or an optical isomer, cis-trans isomer, or a pharmaceutically acceptable salt thereof, in the preparation of an antibiotic synergist: 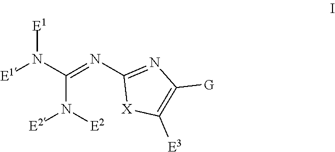
wherein:
E¹ and E¹ are each independently a hydrogen, acyl, substituted or unsubstituted C₁₋₆ alkyl, or substituted or unsubstituted C₁₋₆ alkoxy;
E² and E^{2′} are each independently a hydrogen, acyl, substituted or unsubstituted C₁₋₆ alkyl, or substituted or unsubstituted C₁₋₆ alkoxy; or
one of E¹ and E¹ is connected with one of E² and E^{2′} to form a substituted or unsubstituted C₁₋₆ alkylene;
E³ is a hydrogen, substituted or unsubstituted C₁₋₆ alkyl, substituted or unsubstituted C₁₋₆ alkoxy, substituted or unsubstituted C₃₋₇ cycloalkyl, or halogen;
X is NE⁴, O, S, or Se; E⁴ is a hydrogen, halogen, substituted or unsubstituted C₁₋₃ alkyl, or substituted or unsubstituted C₁₋₃ alkoxy;
G is a substituted or unsubstituted C₁₋₆ alkyl, substituted or unsubstituted C₂₋₆ alkenyl, substituted or unsubstituted C₂₋₆ alkynyl, substituted or unsubstituted C₃₋₇ cycloalkyl, substituted or unsubstituted C₅₋₇ cycloalkenyl, substituted or unsubstituted C₆₋₁₀ aryl (for example, phenyl or naphthyl), substituted or unsubstituted 5- or 6-membered heterocyclic group, or substituted or unsubstituted 8- to 14-membered heteroaryl bicyclic or tricyclic ring system; and the “substituted” refers to a substitution by one or more groups selected from: a halogen, cyano, nitro, R′, OR′, Si(R′)₃, NR′R″, C(O)R′, C(O)OR′, C(O)NR′R″, SR′, S(O)ₘR¹, S(O)₂NR′R″, OC(O)R¹, OC(O)NR′R″, OS(O)₂R¹, OS(O)₂NR′R″, N(R²)C(O)R¹, N(R²)C(O)NR′R″, N(R²)S(O)₂R¹ or N(R²)S(O)₂NR′R″;
wherein R′ and R″ are each independently H, C₁₋₆ alkyl, C₁₋₆ haloalkyl, C₂₋₆ alkenyl, C₂₋₆ haloalkenyl, C₂₋₆ alkynyl, C₂₋₆ haloalkynyl, or substituted or unsubstituted C₃₋₇ cycloalkyl, substituted or unsubstituted C₅₋₇ cycloalkenyl, substituted or unsubstituted phenyl, substituted or unsubstituted naphthyl, substituted or unsubstituted 5- or 6-membered heterocyclyl, or substituted or unsubstituted 8- to 12-membered heteroaryl bicyclic ring system, and the “substituted” refers to a substitution by one or more groups selected from: a halogen, cyano, nitro, C₁₋₆ alkyl, C₁₋₆ haloalkyl, C₂₋₆ alkenyl, C₂₋₆ haloalkenyl, C₂₋₆ alkynyl, C₂₋₆ haloalkynyl, hydroxy, hydroxy-C₁₋₄ alkyl, OR³, NR³R⁴, C(O)R³, C(O)OR³, C(O)NR³R⁴, SR³, S(O)ₘR⁵, S(O)₂NR³R⁴, OC(O)R⁵, OC(O)NR³R⁴, OS(O)₂R⁵, OS(O)₂NR³R⁴, N(R⁶)C(O)R⁵, N(R⁶)C(O)NR³R⁴, N(R⁶)S(O)₂R⁵ or N(R⁶)S(O)₂NR³R⁴;
R¹ is C₁₋₆ alkyl, C₁₋₆ haloalkyl, C₂₋₆ alkenyl, C₂₋₆ haloalkenyl, C₂₋₆ alkynyl, C₂₋₆ haloalkynyl, or substituted or unsubstituted C₃₋₇ cycloalkyl, substituted or unsubstituted C₅₋₇ cycloalkenyl, substituted or unsubstituted phenyl, substituted or unsubstituted naphthyl, substituted or unsubstituted 5- or 6-membered heterocyclyl, or substituted or unsubstituted 8- to 12-membered heteroaryl bicyclic ring system; and the “substituted” refers to a substitution by one or more groups selected from: a halogen, cyano, nitro, C₁₋₆ alkyl, C₁₋₆ haloalkyl, C₂₋₆ alkenyl, C₂₋₆ haloalkenyl, C₂₋₆ alkynyl, and C₂₋₆ haloalkynyl, OR³, NR³R⁴, C(O)R³, C(O)OR³, C(O)NR³R⁴, SR³, S(O)ₘR⁵, S(O)₂NR³R⁴, OC(O)R⁵, OC(O)NR³R⁴, OS(O)₂R⁵, OS(O)₂NR³R⁴, N(R⁶)C(O)R⁵, N(R⁶)C(O)NR³R⁴, N(R⁶)S(O)₂R⁵ or N(R⁶)S(O)₂NR³R⁴;
R² is H, C₁₋₆ alkyl, C₁₋₆ haloalkyl, C₂₋₆ alkenyl, C₂₋₆ haloalkenyl, C₂₋₆ alkynyl or C₂₋₆ haloalkynyl, or substituted or unsubstituted C₃₋₇ cycloalkyl, substituted or unsubstituted C₅₋₇ cycloalkenyl, substituted or unsubstituted phenyl, substituted or unsubstituted naphthyl, substituted or unsubstituted 5- or 6-membered heterocyclic group, or substituted or unsubstituted 8- to 12-membered heteroaryl bicyclic ring system; and the “substituted” refers to a substitution by one or more groups selected from: a halogen, cyano, nitro, C₁₋₆ alkyl, C₁₋₆ haloalkyl, C₂₋₆ alkenyl, C₂₋₆ haloalkenyl, C₂₋₆ alkynyl, C₂₋₆ haloalkynyl, OR³, NR³R⁴, C(O)R³, C(O)OR³, C(O)NR³R⁴, SR³, S(O)ₘR⁵, S(O)₂NR³R⁴, OC(O)R⁵, OC(O)NR³R⁴, OS(O)₂R⁵, OS(O)₂NR³R⁴, N(R⁶)C(O)R⁵, N(R⁶)C(O)NR³R⁴, N(R⁶)S(O)₂R⁵ or N(R⁶)S(O)₂NR³R⁴;
wherein R³, R⁴ and R⁶ are each independently H, C₁₋₆ alkyl, C₁₋₆ haloalkyl, C₂₋₆ alkenyl, C₂₋₆ haloalkenyl, C₂₋₆ alkynyl, or C₂₋₆ haloalkynyl;
R⁵ is C₁₋₆ alkyl, C₁₋₆ haloalkyl, C₂₋₆ alkenyl, C₂₋₆ haloalkenyl, C₂₋₆ alkynyl, or C₂₋₆ haloalkynyl;
and m is 1 or 2.

2. The compound of claim 1, wherein the compound of Formula I is as shown in Formula I′: 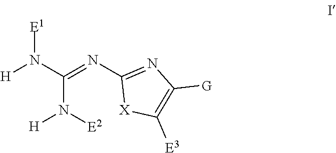
wherein E¹, E², E³, X and G are described as in claim 1.

3. The compound of claim 2, wherein the compound of Formula I′ is as shown in Formula II-a or II-b: 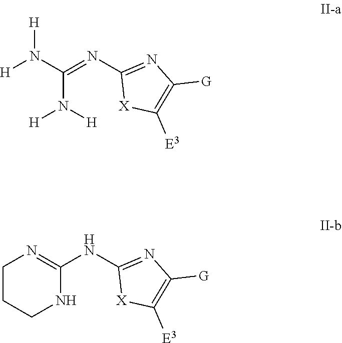
wherein E³, X and G are described as in claim 1.

4. The compound of claim 3, wherein the compound of Formula II-a or II-b is as shown in Formula III-a or III-b: 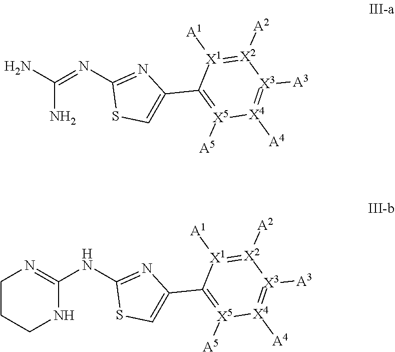
wherein each of X¹, X², X³, X⁴ and X⁵ is independently selected from C or N;
each of A¹, A², A³, A⁴ and A⁵ is independently selected from H, substituted or unsubstituted C₁₋₆ alkyl, substituted or unsubstituted C₁₋₆ haloalkyl, substituted or unsubstituted C₂₋₆ alkenyl, substituted or unsubstituted C₂₋₆ haloalkenyl, substituted or unsubstituted C₂₋₆ alkynyl, substituted or unsubstituted C₂₋₆ haloalkynyl, substituted or unsubstituted C₃₋₇ cycloalkyl, substituted or unsubstituted C₅₋₇ cycloalkenyl, substituted or unsubstituted phenyl, substituted or unsubstituted 5- or 6-membered heterocyclyl; and the “substituted” is the same as those for G as described in claim 1.

5. The compound of claim 4, wherein the compound of Formula III-a or III-b is as shown in Formula IV-a or IV-b: 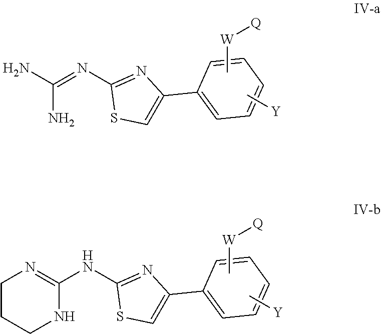
wherein W is selected from: —O(CH₂)ₚ—, —S(CH₂)ₚ—, —CH₂—, —CO—, —S(O)ₚ—, —C(O)NH—, —S(O)₂NH—, —NHC(O)—, Oxime ether group, Thiocarbonyl-NH—; preferably —O(CH₂)ₚ—, —S(CH₂)ₚ—;
p is 0, 1 or 2;
Q is selected from a substituted or unsubstituted C₁₋₆ alkyl, substituted or unsubstituted C₂₋₆ alkenyl, substituted or unsubstituted C₂₋₆ alkynyl, substituted or unsubstituted C₃₋₇ cycloalkyl, substituted or unsubstituted C₅₋₇ cycloalkenyl, substituted or unsubstituted phenyl, substituted or unsubstituted 5- or 6-membered heterocyclyl; and the “substituted” is the same as those for G as described in claim 1;
Y is selected from a hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₇ alkylene, substituted or unsubstituted C₅₋₇ cycloalkenyl, substituted or unsubstituted phenyl, substituted or unsubstituted naphthyl, substituted or unsubstituted 5- or 6-membered heterocyclic group; and the “substituted” is the same as those for G as described in claim 1.

6. The compound of claim 5, wherein Q in the Formula III-a or III-b is as shown in Formula V: 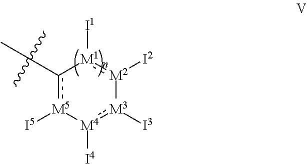
wherein M¹ is selected from C, CH, S, O or N;
each of M², M³, M⁴ and M⁵ is independently selected from: C, CsH, S, O, N;
n is an integer selected from 0-2;
each of I¹, I², I³, I⁴ and I⁵ is independently selected from a hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₇ alkylene, substituted or unsubstituted C₅₋₇ cycloalkenyl, substituted or unsubstituted phenyl, substituted or unsubstituted 5- or 6-membered heterocyclic group; and the “substituted” is the same as those for G as described in claim 1.

7. The compound of claim 1, wherein E¹, E^{1′}, E² and E^{2′} are H; or
one of E¹ and E^{1′} is connected with one of E² and E^{2′} to form a substituted or unsubstituted C₂₋₄ alkylene;
E³ is a hydrogen, substituted or unsubstituted C₁₋₆ alkyl or halogen;
X is NH, O, S or Se;
G is selected from a substituted or unsubstituted phenyl, or substituted or unsubstituted 8- to 14-membered heteroaryl bicyclic ring system; and the “substituted” is described as in claim 1.

8. A compound, or an optical isomer, cis-trans isomer, or a pharmaceutically acceptable salt thereof, in the preparation of an antibiotic synergist, wherein the compound is any one of those numbered I-1 to 1-200;
preferably, the compound is any one of those numbered as follows:
I-9, I-14, I-19, I-50, I-60, I-75, I-81, I-82, I-86, I-86, I-87, I-88, I-89, I-90, I-92, I-93, I-95, I-96, I-97, I-98, I-99, I-100, I-108, I-123, I-124, I-125, I-126, I-127, I-128, I-129, I-130, 1-131, I-132, I-133, I-142, I-143, I-144, I-145, I-146, I-147, I-148, I-149, I-150, I-151, I-152, I-153, I-154, I-155, I-156, I-157, I-158, I-159, I-160, I-161, I-162, I-163, I-170, I-176.

9. The compound of claim 1, wherein the antibiotic is an anti-Gram-negative bacterial antibiotic or a polypeptide antibiotic.

10. A pharmaceutical composition comprising:
a. the compound of claim 1, or an optical isomer, cis-trans isomer, or a pharmaceutically acceptable salt thereof, or a combination thereof; and
b. an antibiotic; and
c. optionally, a pharmaceutically acceptable carrier and/or excipient.
